(19) United States
(12) Patent Application Publication
(10) Pub. No.: US 2018/0311245 A1
(43) Pub. Date: Nov. 1, 2018

(54) USE OF A COMBINATIONAL THERAPY OF LSD1 INHIBITORS WITH CDK2 INHIBITORS IN THE TREATMENT OF CANCER

(71) Applicant: Rasna Therapeutics Limited, London (GB)

(72) Inventor: Kunwar SHAILUBHAI, Audubon, PA (US)

(21) Appl. No.: 15/962,492

(22) Filed: Apr. 25, 2018

Related U.S. Application Data
(60) Provisional application No. 62/490,555, filed on Apr. 26, 2017.

(51) Int. Cl. A61K 31/519; A61P 35/02

(52) U.S. Cl. CPC A61K 31/519; A61K 31/135; A61K 2300/00; A61P 35/02

(57) ABSTRACT

The present disclosure relates to methods of treating cancer and pharmaceutical compositions for use in the treatment of cancer, including LSD1-inhibitor-resistant cancers, comprising administering to a subject an effective amount of a CDK2 inhibitor and an effective amount of a LSD1 inhibitor.

### RELATED APPLICATIONS

This application claims priority to U.S. application Ser. No. 62/490,555, filed Apr. 26, 2017, which is incorporated by reference in its entirety.

### TECHNICAL FIELD

The present disclosure relates to methods of treating cancer comprising administering to a subject an effective amount of a CDK2 inhibitor and an effective amount of a LSD1 inhibitor.

### BACKGROUND

It is estimated that 8.8 million people worldwide died from cancer in 2015, which accounts for nearly one out of every six deaths globally. The World Health Organization estimated that the total economic costs of cancer in 2010 were about $1.16 trillion. (See http://www.who.int/cancer/en/.) As such, there is a need for the development of novel therapies for the treatment of cancers, and in particular, cancers that are resistant to currently available therapies.

### SUMMARY

In one aspect, this application pertains to a method for treating a subject having cancer, which comprises

i. administering to the subject an effective amount of a CDK2 inhibitor; and
ii. administering to the subject an effective amount of a LSD1 inhibitor.

In one embodiment, the CDK2 inhibitor is administered before, concurrently, or after the LSD1 inhibitor.

In one embodiment, the CDK2 inhibitor is administered before the LSD1 inhibitor.

In one embodiment, the CDK2 inhibitor is administered about 24 hours, about 48 hours, about 72 hours, or about 1 week before the LSD1 inhibitor.

In one embodiment, the CDK2 inhibitor is administered concurrently with the LSD1 inhibitor.

In one embodiment, the CDK2 inhibitor is administered after the LSD1 inhibitor.

In one embodiment, the CDK2 inhibitor is selected from the group consisting of: Milciclib, Dinaciclib, AG-024322, 2-Hydroxybohemine, Aloisine A, AZD 5438, BMS-265246, Butyrolactone I, CDK2 Inhibitor II (CAS # 222035-13-4), CDK2 Inhibitor IV, NU6140 (CAS # 444723-13-1), CYC-065, NU2058 (CAS # 161058-83-9), NU6102 (CAS # 444722-95-6), Olomoucine, PHA-793887 (CAS # 718630-59-2), Roscovitine (seliciclib), SCH 900776 (CAS # 891494-63-6), SNS-032 (BMS-387032), SU9516 (CAS # 377090-84-1), WHI-P180 (CAS # 21 1555-08-7), or any pharmaceutically acceptable salt thereof, and any combination thereof.

In one embodiment, the CDK2 inhibitor is milciclib or a pharmaceutically acceptable salt thereof.

In one embodiment, the LSD1 inhibitor is selected from the group consisting of a compound of Formula (I), Formula (Ia), Formula (II), Formula (III), any other compound indicated as an LSD1 inhibitor, or a stereoisomer or a pharmaceutically acceptable salt thereof

In one embodiment, the LSD1 inhibitor is selected from the group consisting of: MC2580, DDP38003, tranylcypromine, (R)-4-[5-(Pyrrolidin-3-ylmethoxy)-2-p-tolyl-pyridin-3-yl]-benzonitrile, 1-(4-methyl-1-piperazinyl)-2-[[(1R*,2S*)-2-[4-phenylmethoxy)phenyl]cyclopropyl]amino]ethanone, N-[4-[trans-2-aminocyclopropyl]phenyl]-4-(4-methylpiperazin-1 -yl)benzamide,

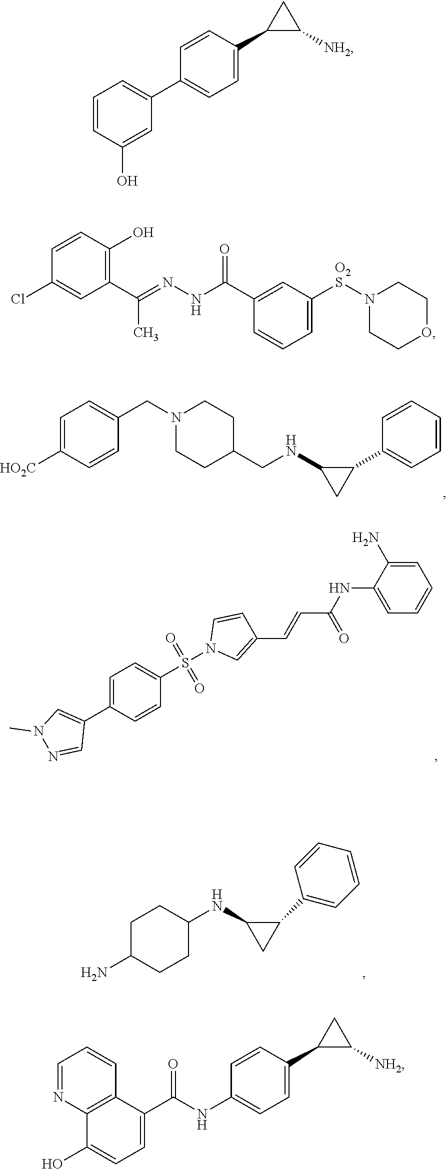

or a stereoisomer or a pharmaceutically acceptable salt thereof.

In one embodiment, the LSD1 inhibitor is MC2580 or a stereoisomer or a pharmaceutically acceptable salt thereof

In one embodiment, the LSD1 inhibitor is DDP38003 or a stereoisomer or a pharmaceutically acceptable salt thereof

In one embodiment, the cancer is selected from the group consisting of leukemia (including acute promyelocytic leukemia and acute myeloid leukemia), prostate cancer, breast cancer, lung cancer (including small cell lung cancer), colorectal cancer, melanomas, bladder cancer, brain/CNS cancer, cervical cancer, esophageal cancer, gastric cancer, head/neck cancer, kidney cancer, liver cancer, lymphomas, ovarian cancer, pancreatic cancer, and sarcomas.

In one embodiment, the cancer is selected from the group consisting of acute promyelocytic leukemia, acute myeloid leukemia, small cell lung carcinoma, and melanoma.

In one embodiment, the cancer is acute promyelocytic leukemia.

In one embodiment, the cancer is acute myeloid leukemia.

In one embodiment, the cancer is small cell lung carcinoma.

In one embodiment, the cancer is melanoma.

In one embodiment, the cancer is a solid tumor or blood tumor.

In one embodiment, the cancer is a solid tumor.

In one embodiment, the cancer is a blood tumor.

In one embodiment, the cancer is a LSD1-inhibitor-resistant cancer.

In one embodiment, the LSD1-inhibitor-resistant cancer comprises cancerous cells having a reduced level of p21 expression or a loss of p21 function as compared to cancerous cells that are sensitive to LSD1 inhibitors.

In one aspect, this application pertains to a method for treating a subject having LSD1-inhibitor-resistant cancer by sensitizing cells of the cancer to LSD1 inhibitors, which comprises

i. administering to the subject an effective amount of a CDK2 inhibitor; and
ii. administering to the subject an effective amount of a LSD1 inhibitor.

In one embodiment, the method for treating a subject having LSD1-inhibitor-resistant cancer by sensitizing cells of the cancer to LSD1 inhibitors additionally comprises a step of evaluating the cancer to predict resistance to LSD1 inhibitors prior to administration of the CDK2 inhibitor.

In one embodiment, for the method for treating a subject having LSD1-inhibitor-resistant cancer by sensitizing cells of the cancer to LSD1 inhibitors, the CDK2 inhibitor is administered before, concurrently, or after the LSD1 inhibitor.

In one embodiment, for the method for treating a subject having LSD1-inhibitor-resistant cancer by sensitizing cells of the cancer to LSD1 inhibitors, the CDK2 inhibitor is administered before the LSD1 inhibitor.

In one embodiment, for the method for treating a subject having LSD1-inhibitor-resistant cancer by sensitizing cells of the cancer to LSD1 inhibitors, the CDK2 inhibitor is administered about 24 hours, about 48 hours, about 72 hours, or about 1 week before the LSD1 inhibitor.

In one embodiment, for the method for treating a subject having LSD1-inhibitor-resistant cancer by sensitizing cells of the cancer to LSD1 inhibitors, the CDK2 inhibitor is administered concurrently with the LSD1 inhibitor.

In one embodiment, for the method for treating a subject having LSD1-inhibitor-resistant cancer by sensitizing cells of the cancer to LSD1 inhibitors, the CDK2 inhibitor is administered after the LSD1 inhibitor.

In one embodiment, the CDK2 inhibitor is selected from the group consisting of: Milciclib, Dinaciclib, AG-024322, 2-Hydroxybohemine, Aloisine A, AZD 5438, BMS-265246, Butyrolactone I, CDK2 Inhibitor II (CAS # 222035-13-4), CDK2 Inhibitor IV, NU6140 (CAS # 444723-13-1), CYC-065, NU2058 (CAS # 161058-83-9), NU6102 (CAS # 444722-95-6), Olomoucine, PHA-793887 (CAS # 718630-59-2), Roscovitine (seliciclib), SCH 900776 (CAS # 891494-63-6), SNS-032 (BMS-387032), SU9516 (CAS # 377090-84-1), WHI-P180 (CAS # 21 1555-08-7), or any pharmaceutically acceptable salt thereof, and any combination thereof.

In one embodiment, the CDK2 inhibitor is milciclib or a pharmaceutically acceptable salt thereof.

In one embodiment, the LSD1 inhibitor is selected from the group consisting of a compound of Formula (I), Formula (Ia), Formula (II), Formula (III), any other compound indicated as an LSD1 inhibitor, or a stereoisomer or a pharmaceutically acceptable salt thereof

In one embodiment, the LSD1 inhibitor is selected from the group consisting of: MC2580, DDP38003, tranylcypromine, (R)-4-[5-(Pyrrolidin-3 -ylmethoxy)-2-p-tolyl-pyridin-3 -yl]-benzonitrile, 1-(4-methyl-1-piperazinyl)-2-[[(1R*,2S*)-2-[4-phenylmethoxy)phenyl]cyclopropyl]amino]ethanone, N-[4-[trans-2-aminocyclopropyl]phenyl]-4-(4-methylpiperazin-1 -yl)benzamide,

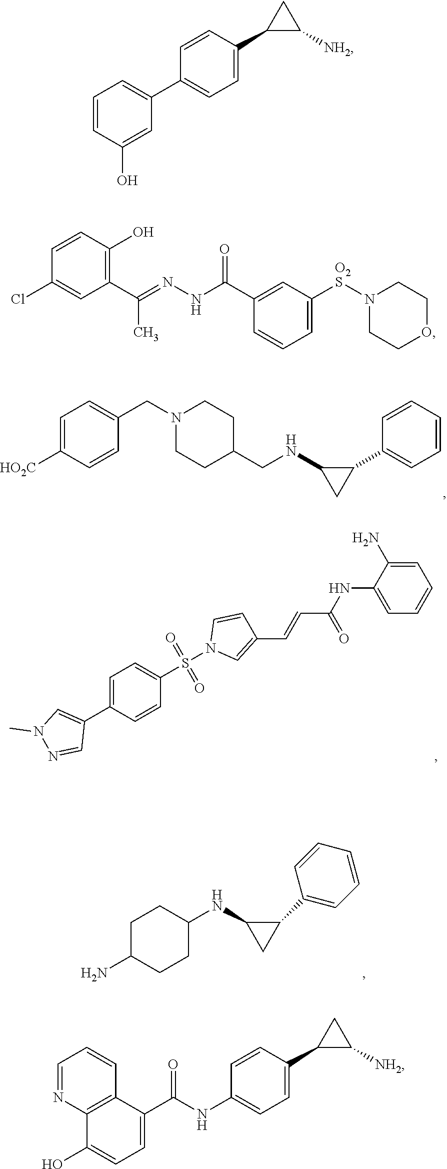

or a stereoisomer or a pharmaceutically acceptable salt thereof.

In one embodiment, the LSD1 inhibitor is MC2580 or a stereoisomer or a pharmaceutically acceptable salt thereof

In one embodiment, the LSD1 inhibitor is DDP38003 or a stereoisomer or a pharmaceutically acceptable salt thereof

In one embodiment, for the method for treating a subject having LSD1-inhibitor-resistant cancer by sensitizing cells of the cancer to LSD1 inhibitors, the LSD1-inhibitor-resistant cancer comprises cancerous cells having a reduced level of p21 expression or a loss of p21 function as compared to cancerous cells that are sensitive to LSD1 inhibitors.

In one embodiment, for the method for treating a subject having LSD1-inhibitor-resistant cancer by sensitizing cells of the cancer to LSD1 inhibitors, the LSD1-inhibitor-resistant cancer comprises cancerous cells having a reduced level of p21 expression as compared to cancerous cells that are sensitive to LSD1 inhibitors.

In one embodiment, for the method for treating a subject having LSD1-inhibitor-resistant cancer by sensitizing cells of the cancer to LSD1 inhibitors, the LSD1-inhibitor-resistant cancer comprises cancerous cells having a loss of p21 function as compared to cancerous cells that are sensitive to LSD1 inhibitors.

In one embodiment, the cancer is selected from the group consisting of leukemia (including acute promyelocytic leukemia and acute myeloid leukemia), prostate cancer, breast cancer, lung cancer (including small cell lung cancer), colorectal cancer, melanomas, bladder cancer, brain/CNS cancer, cervical cancer, esophageal cancer, gastric cancer, head/neck cancer, kidney cancer, liver cancer, lymphomas, ovarian cancer, pancreatic cancer, and sarcomas.

In one embodiment, the cancer is selected from the group consisting of acute promyelocytic leukemia, acute myeloid leukemia, small cell lung carcinoma, and melanoma.

In one embodiment, the cancer is acute promyelocytic leukemia.

In one embodiment, the cancer is acute myeloid leukemia.

In one embodiment, the cancer is small cell lung carcinoma.

In one embodiment, the cancer is melanoma.

In one embodiment, the cancer is a solid tumor or blood tumor.

In one embodiment, the cancer is a solid tumor.

In one embodiment, the cancer is a blood tumor.

In one aspect, this application pertains to a pharmaceutical composition for treating or ameliorating the effects of a cancer in a subject in need thereof, comprising

i. a therapeutically-effective amount of an LSD1 inhibitor;
ii. a therapeutically-effective amount of a CDK2 inhibitor; and
iii. a pharmaceutically-acceptable excipient.

In one embodiment, the pharmaceutical composition for treating or ameliorating the effects of a cancer in a subject in need thereof, the LSD1 inhibitor and the CDK2 inhibitor are in a unified dosage form or in separate dosage forms.

In one embodiment, the pharmaceutical composition for treating or ameliorating the effects of a cancer in a subject in need thereof, the LSD1 inhibitor and the CDK2 inhibitor are in a unified dosage form.

In one embodiment, the pharmaceutical composition for treating or ameliorating the effects of a cancer in a subject in need thereof, the LSD1 inhibitor and the CDK2 inhibitor are in a separate dosage form.

In one embodiment, the pharmaceutical composition for treating or ameliorating the effects of a cancer in a subject in need thereof, the LSD1 inhibitor and the CDK2 inhibitor are co-administered to the subject.

In one embodiment, the pharmaceutical composition for treating or ameliorating the effects of a cancer in a subject in need thereof, the LSD1 inhibitor and the CDK2 inhibitor are administered to the subject serially.

In one embodiment, the pharmaceutical composition for treating or ameliorating the effects of a cancer in a subject in need thereof, the LSD1 inhibitor and the CDK2 inhibitor, the CDK2 inhibitor is administered about 24 hours, about 48 hours, about 72 hours, or about 1 week before the LSD1 inhibitor.

In one embodiment, the pharmaceutical composition for treating or ameliorating the effects of a cancer in a subject in need thereof, the LSD1 inhibitor and the CDK2 inhibitor, the LSD1 inhibitor is administered about 24 hours, about 48 hours, about 72 hours, or about 1 week before the CDK2 inhibitor.

In one embodiment, the pharmaceutical composition for treating or ameliorating the effects of a cancer in a subject in need thereof, the CDK2 inhibitor is selected from the group consisting of: Milciclib, Dinaciclib, AG-024322, 2-Hydroxybohemine, Aloisine A, AZD 5438, BMS-265246, Butyrolactone I, CDK2 Inhibitor II (CAS # 222035-13-4), CDK2 Inhibitor IV, NU6140 (CAS # 444723-13-1), CYC-065, NU2058 (CAS # 161058-83-9), NU6102 (CAS # 444722-95-6), Olomoucine, PHA-793887 (CAS # 718630-59-2), Roscovitine (seliciclib), SCH 900776 (CAS # 891494-63-6), SNS-032 (BMS-387032), SU9516 (CAS # 377090-84-1), WHI-P180 (CAS # 21 1555-08-7), or any pharmaceutically acceptable salt thereof, and any combination thereof.

In one embodiment, the pharmaceutical composition for treating or ameliorating the effects of a cancer in a subject in need thereof, the CDK2 inhibitor is milciclib or a pharmaceutically acceptable salt thereof.

In one embodiment, the pharmaceutical composition for treating or ameliorating the effects of a cancer in a subject in need thereof, the LSD1 inhibitor is selected from the group consisting of a compound of Formula (I), Formula (Ia), Formula (II), Formula (III), any other compound indicated as an LSD1 inhibitor, or a stereoisomer or a pharmaceutically acceptable salt thereof.

In one embodiment, the pharmaceutical composition for treating or ameliorating the effects of a cancer in a subject in need thereof, the LSD1 inhibitor is selected from the group consisting of: MC2580, DDP38003, tranylcypromine, (R)-4-[5-(Pyrrolidin-3-ylmethoxy)-2-p-tolyl-pyridin-3-yl]-benzonitrile, 1-(4-methyl-1-piperazinyl)-2-[[(1R*,2S*)-2-[4-phenylmethoxy)phenyl]cyclopropyl]amino]ethanone, N-[4-[trans-2-aminocyclopropyl]phenyl]-4-(4-methylpiperazin-1-yl)benzamide,

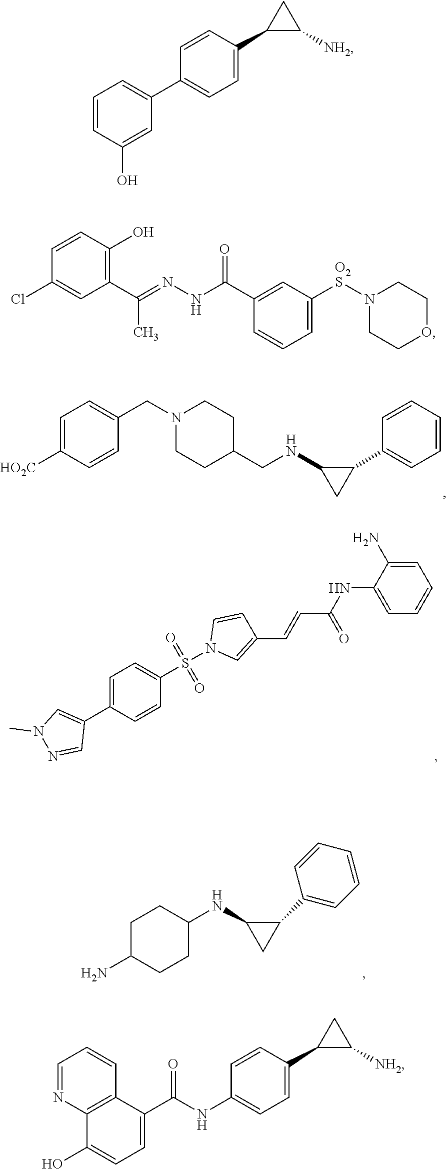

or a stereoisomer or a pharmaceutically acceptable salt thereof.

In one embodiment, the pharmaceutical composition for treating or ameliorating the effects of a cancer in a subject in need thereof, the LSD1 inhibitor is MC2580 or a stereoisomer or a pharmaceutically acceptable salt thereof.

In one embodiment, the pharmaceutical composition for treating or ameliorating the effects of a cancer in a subject in need thereof, the LSD1 inhibitor is DDP38003 or a stereoisomer or a pharmaceutically acceptable salt thereof.

In one embodiment, the pharmaceutical composition for treating or ameliorating the effects of a cancer in a subject in need thereof, the cancer is selected from the group consisting of leukemia (including acute promyelocytic leukemia and acute myeloid leukemia), prostate cancer, breast cancer, lung cancer (including small cell lung cancer), colorectal cancer, melanomas, bladder cancer, brain/CNS cancer, cervical cancer, esophageal cancer, gastric cancer, head/neck cancer, kidney cancer, liver cancer, lymphomas, ovarian cancer, pancreatic cancer, and sarcomas.

In one embodiment, the cancer is selected from the group consisting of acute promyelocytic leukemia, acute myeloid leukemia, small cell lung carcinoma, and melanoma.

In one embodiment, the cancer is acute promyelocytic leukemia.

In one embodiment, the cancer is acute myeloid leukemia.

In one embodiment, the cancer is small cell lung carcinoma.

In one embodiment, the cancer is melanoma.

In one embodiment, the cancer is a solid tumor or blood tumor.

In one embodiment, the cancer is a solid tumor.

In one embodiment, the cancer is a blood tumor.

In one embodiment, the cancer is a LSD1-inhibitor-resistant cancer.

In one embodiment, the pharmaceutical composition for treating or ameliorating the effects of a cancer in a subject in need thereof, the LSD1-inhibitor-resistant cancer comprises cancerous cells having a reduced level of p21 expression or a loss of p21 function as compared to cancerous cells that are sensitive to LSD1 inhibitors.

In one embodiment, the pharmaceutical composition for treating or ameliorating the effects of a cancer in a subject in need thereof, the LSD1-inhibitor-resistant cancer comprises cancerous cells having a reduced level of p21 expression as compared to cancerous cells that are sensitive to LSD1 inhibitors.

In one embodiment, the pharmaceutical composition for treating or ameliorating the effects of a cancer in a subject in need thereof, the LSD1-inhibitor-resistant cancer comprises cancerous cells having a loss of p21 function as compared to cancerous cells that are sensitive to LSD1 inhibitors.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Other methods and features will become apparent to those skilled in the art upon examination of the following drawings and detailed description. It is intended that all such methods and features be included within this description, be within the scope of the present invention, and be protected by the accompanying claims.

### DETAILED DESCRIPTION

In the present application, the term “CDK2 inhibitor” refers to a compound that inhibits the enzyme in humans referred to cyclin-dependent kinase (CDK) 2.

The p21 gene encodes a cyclin dependent kinase inhibitor which affects cell cycle progression. Expression of p21 resulted in an accumulation of cells in G0/G1, alteration in morphology, and cell differentiation.

In the present application the LSD1 inhibitor is any known LSD1 inhibitor, for instance an LSD1 inhibitor as described in WO2013057322, WO2011131576, WO2014086790, WO2012135113, WO2015/181380 and WO2016/34946, each of which are incorporated herein by reference in their entireties.

In one embodiment, the LSD1 inhibitor may also be an antisense, an antibody, or a monoclonal antibody.

In one embodiment, the LSD1 inhibitor may also be referred to as a KDM1A inhibitor

In one embodiment, the LSD1 inhibitor is a compound referred to herein as DDP 38003, DDP38003, DDP-38003 (CAS No. 1831167-97-5), or any pharmaceutically acceptable salt thereof

The structure of the compound referred to as DDP38003 is:

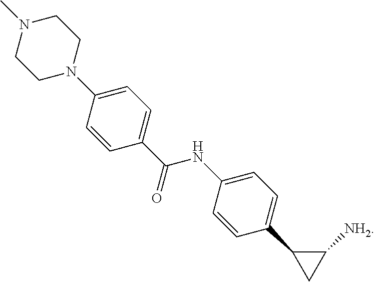 [SMILES: CN1CCN(c2ccc(C(=O)Nc3ccc([C@@H]4C[C@H]4N)cc3)cc2)CC1]

In one embodiment, the LSD1 inhibitor is a compound that is disclosed in Binda et al. in J. Am. Chem. Soc. 2010, 132, 6827-6833, which is incorporated by reference herein in its entirety.

In one embodiment, the LSD1 inhibitor is a compound referred to herein as MC 2580, MC2580, MC-2580, or any pharmaceutically acceptable salt thereof. The structure of the compound

referred to as MC2580 is:

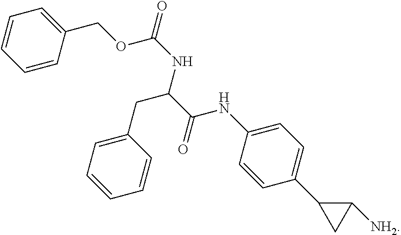 [SMILES: NC1CC1c1ccc(NC(=O)C(Cc2ccccc2)NC(=O)OCc2ccccc2)cc1]

In one embodiment the LSD1 inhibitor is selected from a compound of Formula (I)

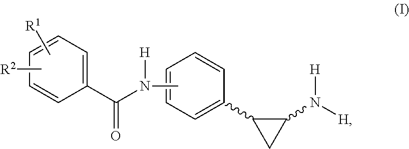

or a pharmaceutically acceptable salt, solvate, or prodrug thereof, wherein:
R¹ is heterocyclyl or heterocyclyl substituted by oxo, wherein the heterocyclyl is unsubstituted or substituted by one or more C₁-C₆ alkyl;
R² is hydrogen, halogen, C₁-C₆ alkyl, C₁-C₆ alkoxy, C₁-C₆ haloalkyl, C₁-C₆ haloalkoxy, or benzyloxycarbonylamino.

In one embodiment, the LSD1 inhibitor is selected from a compound of Formula (I) that is: N[4-[trans-2-aminocyclopropyl]phenyl]-4-(4-methylpiperazin-1-yl)benzamide; N-[4-[trans-2-aminocyclopropyl]phenyl]-4-(1-methyl-4-piperidyl)benzamide; N-[4-[trans-2-aminocyclopropyl]phenyl]-3-(2-oxooxazolidin-3-yl)benzamide; N-[4-[trans-2-aminocyclopropyl]phenyl]-4-morpholino-benzamide; N-[4-[trans-2-aminocyclopropyl]phenyl]-4-(2-oxooxazolidin-3-yl)benzamide; benzyl N-[5-[[4-[(trans-2-aminocyclopropyl]phenyl]carbamoyl]-2-(4-methylpiperazin-1-yl)phenyl]carbamate; benzyl N-[4-[[4-[trans-2-aminocyclopropyl]phenyl]carbamoyl]-2-(4-methylpiperazin-1-yl)phenyl]carbamate; benzyl N-[5-[[4-[trans-2-aminocyclopropyl]phenyl]carbamoyl]-2-(1-piperidyl)phenyl]carbamate; benzyl N-[5-[[4-[trans-2-aminocyclopropyl]phenyl]carbamoyl]-2-morpholino-phenyl]carbamate; N-[4-[(1S,2R)-2-aminocyclopropyl]phenyl]-4-(4-methylpiperazin-1-yl)benzamide; N-[4-[(1S,2R)-2-aminocyclopropyl]phenyl]-3-(2-oxooxazolidin-3-yl)benzamide; N-[4-[(1R,2S)-2-aminocyclopropyl]phenyl]-4-(4-methylpiperazin-1-yl)benzamide; N-[4-[(1R,2S)-2-aminocyclopropyl]phenyl]-3-(2-oxooxazolidin-3-yl)benzamide; or a pharmaceutically acceptable salt, solvate, or prodrug thereof.

In one embodiment the LSD1 inhibitor is selected from a compound of Formula (Ia)

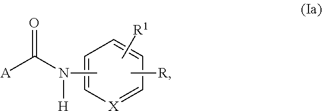

or a stereoisomer or pharmaceutically acceptable salt thereof, wherein:

### A is

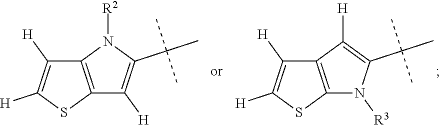

### X is CH or N;

### R is L1-R⁴;

R¹ is H, halogen, C1-C6-alkyl, C1-C6-alkoxy, —CH₂—Z—R⁵, or —Z—CH₂—R⁶;
R² and R³ are C₁-C₄-alkyl;
L1 is −(CH₂)j—Y—, —Y—(CH₂)ₖ-, —CH₂—CH₂— or —CO—NH—;
j and k are, independently, each an integer from 1 to 6;
Y is oxygen, sulphur, NH or N(C₁-C₆-alkyl);
Z is a bond, oxygen, sulphur, NH or N(C₁-C₆-alkyl);
R⁴, R⁵, and R⁶ are, independently, C₁-C₆-alkyl, aryl, heteroaryl, wherein the aryl or heteroaryl are optionally substituted by halogen, C₁-C₆-alkyl, or L2-R⁷; or heterocyclyl, wherein the heterocyclyl is optionally substituted by C₁-C₆-alkyl;
L2 is —(CH₂)ₘ- or —(CH₂)ₙ—W—(CH₂)ₒ-;
R⁷ is C₁-C₆-alkylamino, C₃-C₇ cycloalkyl or heterocyclyl, wherein the C₃-C₇ cycloalkyl or heterocyclyl are optionally substituted by C₁-C₆-alkyl, or NH₂; or guanidine;
m, n, o are, independently, each zero or an integer from 1 to 6;
W is oxygen, sulphur, NH, or CH₂;
wherein aryl is a mono or bicyclic aromatic ring system of 6 or 9 or 10 atoms; heteroaryl is a mono or bicyclic heteroaromatic ring system of 5 to 10 members, which contains one, two, three or four heteroatoms selected from nitrogen, oxygen and sulphur, and one to nine carbon atoms; and
heterocyclyl is a mono, bicyclic or a spirocyclic saturated or partially saturated non-aromatic ring system of 4 to 12 members, which contains one, two, or three heteroatoms selected from nitrogen, oxygen, and sulphur, and three to eleven carbon atoms;

In one embodiment, the LSD1 inhibitor is selected from a compound of Formula (Ia) that is: 4-methyl-N-[2-[[4-(4-piperidyloxy)phenoxy]methyl]phenyl]thieno[3,2-b]pyrrole-5-carboxamide; 4-methyl-N-[2-[[4-(4-piperidylmethoxy)phenoxy]methyl]phenyl]thieno-[3,2-b]pyrrole-5-carboxamide; N-[2-[[4-[(1-ethyl-4-piperidyl)oxy]phenoxy]methyl]phenyl]-4-methyl-thieno[3 ,2-b]pyrrole-5-carboxamide; 4-methyl-N-[2-[[4-[[(3R)-pyrrolidin-3-yl]methoxy]phenoxy]methyl]phenyl]thieno[3,2-b]pyrrole-5-carboxamide; 4-methyl-N-[2-[[4-(pyrrolidin-3-ylmethoxy)phenoxy]methyl]phenyl]thieno[3,2-b]pyrrole-5-carboxamide; 4-ethyl-N-[3-[[4-(4-piperidyloxy)phenoxy]methyl]phenyl]thieno[3,2-b]pyrrole-5-carboxamide; N-[2-[[4-(azepan-4-yloxy)phenoxy]methyl]phenyl]-4-methyl-thieno[3,2-b]pyrrole-5-carboxamide; 4-ethyl-N-[2-[[4-(4-piperidyloxy)phenoxy]methyl]phenyl]thieno[3,2-b]pyrrole-5-carboxamide; N-[2-[[4-(cis-4-aminocyclohexoxy)phenoxy]methyl]phenyl]-4-methyl-thieno[3,2-b]pyrrole-5-carboxamide; 4-methyl-N-[3-[[4-(4-piperidyloxy)phenoxy]methyl]phenyl]thieno[3,2-b]pyrrole-5-carboxamide; 4-methyl-N-[2-[[4-[[(3S)-pyrrolidin-3-yl]methoxy]phenoxy]methyl]phenyl]thieno[3,2-b]pyrrole-5-carboxamide; 4-ethyl-N[2-[[4-[(1-methyl-4-piperidyl)oxy]phenoxy]methyl]phenyl]thieno[3,2-b]pyrrole-5-carboxamide; 4-methyl-N-[2-[[4-[(1-methyl-4-piperidyl)oxy]phenoxy]methyl]phenyl]thieno[3,2-b]pyrrole-5-carboxamide; N-[2-[[4-(trans-4-aminocyclohexoxy)phenoxy]methyl]phenyl]-4-methyl-thieno[3,2-b]pyrrole-5-carboxamide; 4-methyl-N-[3-[[4-[(1-methyl-4-piperidyl)oxy]phenoxy]methyl]phenyl]thieno[3,2-b]pyrrole-5-carboxamide; 4-methyl-N-[2-[(4-pyrrolidin-3-yloxyphenoxy)methyl]phenyl]thieno[3,2-b]pyrrole-5-carboxamide; 4-methyl-N-[2-[[3-[(1-methyl-4-piperidyl)oxy]phenoxy]methyl]phenyl]thieno[3,2-b]pyrrole-5-carboxamide; 4-methyl-N-[2-[[3-(4-piperidyloxy)phenoxy]methyl]phenyl]thieno[3,2-b]pyrrole-5-carboxamide; 4-methyl-N-[3-[(1-methyl-3-piperidyl)methoxy]phenyl]thieno[3,2-b]pyrrole-5-carboxamide; 4-methyl-N-[2-[[4[(4-methylpiperazin-1-yl)methyl]phenoxy]methyl]phenyl]thieno[3,2-b]pyrrole-5-carboxamide; 4-methyl-N-[3-(4-pyridylmethoxy)phenyl]thieno[3,2-b]pyrrole-5-carboxamide; 4-methyl-N-[3-(4-pyridyloxymethyl)phenyl]thieno[3,2-b]pyrrole-5-carboxamide; 4-methyl-N-[4-[[4-[(1-methyl-4-piperidyl)oxy]phenoxy]methyl]phenyl]thieno[3,2-b]pyrrole-5-carboxamide; N-[2-[[4-[[(1S,5R)-8-azabicyclo[3.2.1]octan-3-yl]oxy]phenoxy]methyl]phenyl]-4-methyl-thieno[3,2-b]pyrrole-5-carboxamide; N-[3-[[4-[[(1S,5R)-8-azabicyclo[3.2.1]octan-3-yl]oxy]phenoxy]methyl]phenyl]-4-methyl-thieno[3,2-b]pyrrole-5-carboxamide; 4-methyl-N-[3-[[4-(4-piperidylmethoxy)phenoxy]methyl]phenyl]thieno[3,2-b]pyrrole-5-carboxamide; 4-methyl-N-[2-[[4-(3-piperidyloxy)phenoxy]methyl]phenyl]thieno[3,2-b]pyrrole-5-carboxamide; 4-methyl-N-[3-[[4-(3-piperidyloxy)phenoxy]methyl]phenyl]thieno[3,2-b]pyrrole-5-carboxamide; N-[3-[[4-(trans-4-aminocyclohexoxy)phenoxy]methyl]phenyl]-4-methyl-thieno[3,2-b]pyrrole-5-carboxamide; N-[3-[[4-(azetidin-3-ylmethoxy)phenoxy]methyl]phenyl]-4-methyl-thieno[3,2-b]pyrrole-5-carboxamide; 4-methyl-N-[2-[[4-(3-methylaminopropoxy)phenoxy]methyl]phenyl]thieno[3,2-b]pyrrole-5-carboxamide; 4-methyl-N-[3-[(4-pyrrolidin-3-yloxyphenoxy)methyl]phenyl]thieno[3,2-b]pyrrole-5-carboxamide; N-[3-[[4-(azepan-4-yloxy)phenoxy]methyl]phenyl]-4-methyl-thieno[3,2-b]pyrrole-5-carboxamide; N-[3-[[4-(cis-4-aminocyclohexoxy)phenoxy]methyl]phenyl]-4-methyl-thieno[3,2-b]pyrrole-5-carboxamide; 4-methyl-N-[3-[[4-(pyrrolidin-3-ylmethoxy)phenoxy]methyl]phenyl]thieno[3,2-b]pyrrole-5-carboxamide; 4-methyl-N-[2-[[4-(4-piperidyloxy)phenyl]carbamoyl]phenyl]thieno[3,2-b]pyrrole-5-carboxamide; 4-methyl-N-[2-[(4-piperazin-1-ylphenoxy)methyl]phenyl]thieno[3,2-b]pyrrole-5-carboxamide; 4-methyl-N-[2-[[4-[(1-methyl-4-piperidyl)oxy]phenyl]methoxy]phenyl]thieno[3,2-b]pyrrole-5-carboxamide; 4-methyl-N-[2-[[4-(4-piperidylamino)phenoxy]methyl]phenyl]thieno[3,2-b]pyrrole-5-carboxamide; N-[2-[[4-(azetidin-3-ylmethoxy)phenoxy]methyl]phenyl]-4-methyl-thieno[3,2-b]pyrrole-5-carboxamide; N-[2-[[4-(2,8-diazaspiro[4.5]decan-2-ylmethyl)phenoxy]methyl]phenyl]-4-methyl-thieno[3,2-b]pyrrole-5-carboxamide; N-[3-(methoxymethyl)-2-[[4-[[(3R)-pyrrolidin-3-yl]methoxy]phenoxy]methyl]phenyl]-4-methyl-thieno[3,2-b]pyrrole-5-carboxamide; N-[3-(methoxymethyl)-2-[[4-(4-piperidyloxy)phenoxy]methyl]phenyl]-4-methyl-thieno[3,2-b]pyrrole-5-carboxamide; N-[2-[[4-(azepan-4-yloxy)phenoxy]methyl]-3-(methoxymethyl)phenyl]-4-methyl-thieno[3,2-b]pyrrole-5-carboxamide; N-[3-(methoxymethyl)-2-[[4-[[(3 S)-pyrrolidin-3-yl]methoxy]phenoxy]methyl]phenyl]-4-methyl-thieno[3,2-b]pyrrole-5-carboxamide; N-[3-(ethoxymethyl)-2-[[4-(4-piperidyloxy)phenoxy]methyl]phenyl]-4-methyl-thieno[3,2-b]pyrrole-5-carboxamide; N-[3-(isopropoxymethyl)-2-[[4-(4-piperidyloxy)phenoxy]methyl]phenyl]-4-methyl-thieno[3,2-b]pyrrole-5-carboxamide; N-[3-(ethoxymethyl)-2-[[4-[[(3R)-pyrrolidin-3-yl]methoxy]phenoxy]methyl]phenyl]-4-methyl-thieno[3,2-b]pyrrole-5-carboxamide; 4-ethyl-N-[3-(methoxymethyl)-2-[[4-[[(3R)-pyrrolidin-3-yl]methoxy]phenoxy]methyl]phenyl]thieno[3,2-b]pyrrole-5-carboxamide; 4-methyl-N-[3-(morpholinomethyl)-2-[[4-(4-piperidyloxy)phenoxy]methyl]phenyl]thieno[3,2-b]pyrrole-5-carboxamide; 4-methyl-N-[5-methyl-2-[[4-(pyrrolidin-3-ylmethoxy)phenoxy]methyl]phenyl]thieno[3,2-b]pyrrole-5-carboxamide; 4-methyl-N-[2-[[5-(4-piperidyloxy)-2-pyridyl]oxymethyl]phenyl]thieno[3,2-b]pyrrole-5-carboxamide; 4-methyl-N-[2-(4-piperidylmethoxy)phenyl]thieno[3,2-b]pyrrole-5-carboxamide; 4-methyl-N-[3-[[3-(4-piperidyloxy)phenoxy]methyl]phenyl]thieno[3,2-b]pyrrole-5-carboxamide; N-[3-[[4-(guanidinomethyl)phenoxy]methyl]phenyl]-4-methyl-thieno[3,2-b]pyrrole-5-carboxamide; N-[2-[[4-(guanidinomethyl)phenoxy]methyl]phenyl]-4-methyl-thieno[3,2-b]pyrrole-5-carboxamide; 4-methyl-N-[2-[2-[4-(4-piperidyloxy)phenyl]ethyl]phenyl]thieno[3,2-b]pyrrole-5-carboxamide; 4-methyl-N-[3-[2-[4-(4-piperidyloxy)phenyl]ethyl]phenyl]thieno[3,2-b]pyrrole-5-carboxamide; 4-methyl-N-[2-[[4-(4-piperidyloxy)anilino]methyl]phenyl]thieno[3,2-b]pyrrole-5-carboxamide; 6-methyl-N-[2-[[3-(4-piperidyloxy)phenoxy]methyl]phenyl]thieno[2,3-b]pyrrole-5-carboxamide; 6-ethyl-N-[2-[[3-(4-piperidyloxy)phenoxy]methyl]phenyl]thieno[2,3-b]pyrrole-5-carboxamide; 6-ethyl-N-[2-[[4-(4-piperidyloxy)phenoxy]methyl]phenyl]thieno[2,3-b]pyrrole-5-carboxamide; 6-methyl-N-[2-[[4-(4-piperidyloxy)phenoxy]methyl]phenyl]thieno[2,3-b]pyrrole-5-carboxamide; 6-methyl-N-[2-[[4-(pyrrolidin-3-ylmethoxy)phenoxy]methyl]phenyl]thieno[2,3-b]pyrrole-5-carboxamide; 4-methyl-N-[4-[[4-(4-piperidyloxy)phenyl]carbamoyl]-2-pyridyl]thieno[3,2-b]pyrrole-5-carboxamide; or a stereoisomer or pharmaceutically acceptable salt thereof.

In one embodiment, the LSD1 inhibitor is selected from a compound of Formula (II)

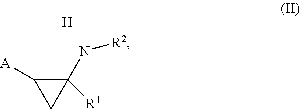

wherein:
A is aryl or heteroaryl, wherein the aryl or heteroaryl may be optionally substituted by one or more substituents independently selected from the group consisting of halogen, C₁-C₆ alkyl, C₁-C₆ alkoxy, C₁-C₆ haloalkyl, C₁-C₆ haloalkoxy, CN, nitro, NH₂, azide, OH, C₁-C₆ alkylamino, and R-L-;
R is aryl, wherein the aryl may be optionally substituted by one, two or more substituents independently selected from the group consisting of halogen, C₁-C₆ alkyl, C₁-C₆ alkoxy, C₁-C₆ haloalkyl, C₁-C₆ haloalkoxy, CN, nitro, NH₂, azide, C₁-C₆ alkylamino optionally substituted by OH, heterocyclylamino optionally substituted by C₁-C₆ alkyl, OH, phenyl, heterocyclyl optionally substituted by C₁-C₆ alkyl, heterocyclyl substituted by oxo, heteroaryl, and benzyloxycarbonylamino; or heteroaryl;
L is a single bond; C₁-C₆ alkylene; C₂-C₆ alkenylene; —(CH₂)ₘX—(CH₂)ₙ—; —(CH₂)ₒ(SO₂)NH—; —(CH₂)ₚ(CO)NR³—; —(CH₂)_{q}NR⁴(CO)—; heterocyclyl substituted by oxo; or heteroaryl;
R¹ is C₁-C₆ alkyl, optionally substituted by aryl or heteroaryl; aryl; heteroaryl; or —(CH₂)ᵣ—Y—R⁵; and
wherein the aryl or heteroaryl group may be optionally substituted by one or more substituents independently selected from the group consisting of halogen, C₁-C₆ alkyl, C₁-C₆ alkoxy, C₁-C₆ haloalkyl, C₁-C₆ haloalkoxy, nitro, acetamido, and phenyl;
R² is hydrogen; C₁-C₆ alkyl, optionally substituted by aryl, heteroaryl, or by heterocyclyl and wherein the aryl or heteroaryl may be optionally substituted by one or more substituents independently selected from the group consisting of halogen, C₁-C₆ alkyl, C₁-C₆ alkoxy, C₁-C₆ haloalkyl, C₁-C₆ haloalkoxy, and NH₂; or —CH₂(CO)NR⁶R⁷;
m, n, o, p, q are, independently, zero or an integer from 1 to 6;
r is an integer from 1 to 6;
X and Y are, independently, NR⁸; O; or S;
R³ and R⁴ are, independently, hydrogen; or C₁-C₆ alkyl;
R⁵ is hydrogen, aryl or heteroaryl, wherein the aryl or heteroaryl may be optionally substituted by one or more substituents independently selected from the group consisting of halogen, C₁-C₆ alkyl, C₁-C₆ alkoxy, C₁-C₆ haloalkyl, C₁-C₆ haloalkoxy, and phenyl;
R⁶ and R⁷ are, independently, hydrogen; C₁-C₆ alkyl; or R⁶ and R⁷ together with the nitrogen to which they are bound form a C₄-C₁₀-heterocyclic ring, optionally containing one or more further heteroatoms in the ring independently selected from NR⁹, O or S and being optionally substituted by NH₂;
R⁸ is hydrogen; C₁-C₆ alkyl, optionally substituted by aryl or heterocyclyl; or C₃₋₆ cycloalkyl;
R⁹ is hydrogen or C₁-C₆ alkyl; or stereoisomers or pharmaceutically acceptable salts thereof.

In one embodiment, the LSD1 inhibitor is selected from a compound of Formula (II) that is: (1 S,2R)- 1 -ethyl-2-phenyl-cyclopropanamine; (1R,2 S)- 1 -ethyl-2-phenyl-cyclopropanamine; trans-1-methyl-2-phenyl-cyclopropanamine; (1R,2 S)- 1-methyl-2-phenyl-cyclopropanamine; (1S,2R)- 1-methyl-2-phenyl-cyclopropanamine; trans-1 -propyl-2-phenyl-cyclopropanamine; trans-1-isopropyl-2-phenyl-cyclopropanamine; trans-1-benzyl-2-phenyl-cyclopropanamine; (1S,2S)-1-benzyl-2-phenyl-cyclopropanamine; (1R,2R)-1-benzyl-2-phenyl-cyclopropanamine; trans-1-phenethyl-2-phenyl-cyclopropanamine; trans-2-(4-bromophenyl)- 1-ethyl-cyclopropanamine; trans- 1-benzyl-2-(4-bromophenyl)cyclopropanamine; trans-1-ethyl-2-(6-quinolyl)cyclopropanamine; trans-1-(2-naphthylmethyl)-2-phenyl-cyclopropanamine; trans-1-ethyl-2-(4-fluorophenyl)cyclopropanamine; trans-1-ethyl-2-(4-chlorophenyl)cyclopropanamine; trans-1-ethyl-2-[3-(trifluoromethyl)phenyl]cyclopropanamine; trans-1-ethyl-2-[4-(trifluoromethyl)phenyl]cyclopropanamine; trans-1-ethyl-2-[3 -fluorophenyl)cyclopropanamine; trans-1-ethyl-2-(3-chlorophenyl)-cyclopropanamine; trans-1 -ethyl-2-(3-bromophenyl)-cyclopropanamine; trans- 1-ethyl-2-[3-methoxyphenyl]cyclopropanamine; 1-ethyl-(trans)-2-[4-(trifluoromethoxy)phenyl]cyclopropanamine; trans-1-ethyl-2-(2-fluorophenyl)cyclopropanamine; trans-1-ethyl-2-(2-chlorophenyl)-cyclopropanamine; trans-1-ethyl-2-(2-bromophenyl)-cyclopropanamine; trans-1-(1-naphthylmethyl)-2-phenyl-cyclopropanamine; trans-2-(4-bromophenyl)-1-(2-naphthylmethyl)cyclopropanamine; trans-N-[4-[2-amino-2-ethyl-cyclopropyl]phenyl]naphthalene-2-carboxamide; N-[2-[(trans)-2-amino-2-ethyl-cyclopropyl]phenyl]benzamide; benzyl N-[3[[2-[(trans)-2-amino-2-ethyl-cyclopropyl]phenyl]carbamoyl]phenyl]carba-mate; benzyl N-[3-[[3-[(trans)-2-amino-2-ethyl-cyclopropyl]phenyl]carbamoyl]phenyl]carba-mate; N-[4-[(trans)-2-amino-2-ethyl-cyclopropyl]phenyl]-3-chloro-benzamide N-[4-[(trans)-2-amino-2-ethyl-cyclopropyl]phenyl]-3-phenyl-benzamide; N-[4-[(trans)-2-amino-2-ethyl-cyclopropyl]phenyl]-4-phenyl-benzamide; N-[3-[[4-[(trans)-2-amino-2-ethyl-cyclopropyl]phenyl]carbamoyl]phenyl]carba-mate; N-[4-[[4-[(trans)-2-amino-2-ethyl-cyclopropyl]phenyl]carbamoyl]phenyl]carba-mate; N-[4-[(trans)-2-amino-2-ethyl-cyclopropyl]phenyl]-2-phenyl-acetamide; N-[4-[(trans)-2-amino-2-ethyl-cyclopropyl]phenyl]-3-phenyl-propanamide; 2-(4-benzyloxyphenyl)-trans-1-ethyl-cyclopropanamine; N-[4-[(2-amino-trans-2-ethyl-cyclopropyl]phenyl]benzenesulfonamide; trans-1-benzyl-2-(4-benzyloxyphenyl)cyclopropanamine; N-[4-[trans-2-amino-2-(2-naphthylmethyl)cyclopropyl]phenyl]benzamide; benzyl-N-[3-[[4-[(trans)-2-amino-2-(2-naphthylmethyl)cyclopropyl]phenyl]carbamoyl]phenyl]carbamate; N-[4-[(trans)-2-amino-2-(2-naphthylmethyl)cyclopropyl]phenyl]-2-phenyl-acetamide; N-[4-[trans-2-amino-2-ethyl-cyclopropyl]phenyl]benzamide; trans-4-(2-amino-2-ethyl-cyclopropyl)aniline; trans-2-(3-azidophenyl)-1-ethyl-cyclopropanamine; 1-amino-(trans)-2-phenyl-cyclopropyl]methanol; 1-amino-(cis)-2-phenyl-cyclopropyl]methanol; (1R,2S)-1-ethyl-N-[(2-methoxyphenyl)methyl]-2-phenyl-cyclopropanamine; (1R,2S)-1-ethyl-N-[(2-methoxy-1-naphthyl)methyl]-2-phenyl-cyclopropanamine; 2-[[(1S,2R)-1-methyl-2-phenyl-cyclopropyl]amino]-1-(4-methylpiperazin-1-yl)ethanone; 2-[[(1S,2S)-1-methyl-2-phenyl-cyclopropyl]amino]-1-(4-methylpiperazin-1-yl)ethanone; 1-[(3S)-3-aminopyrrolidin-1-yl]-2-[[(1S,2R)-1-methyl-2-phenyl-cyclopropyl]amino]ethanone; trans-2-[[(1-ethyl-2-phenyl-cyclopropyl]amino]-1-(4-methylpiperazin-1-yl)ethanone; cis-2-[[(1-ethyl-2-phenyl-cyclopropyl]amino]-1-(4-methylpiperazin-1-yl)ethanone; trans-1-ethyl-N-methyl-2-phenyl-cyclopropanamine; cis-1-ethyl-N-methyl-2-phenyl-cyclopropanamine; trans-1-ethyl-N-ethyl-2-phenyl-cyclopropanamine; cis-1-ethyl-N-ethyl-2-phenyl-cyclopropanamine; trans-2-[[1-ethyl-2-phenyl-cyclopropyl]amino]acetamide; trans-N-benzyl-1-ethyl-2-phenyl-cyclopropanamine; trans-N-[(3,4-dimethoxyphenyl)methyl]-1-ethyl-2-phenyl-cyclopropanamine; trans-N-[(4, 7-dimethoxy-1-naphthyl)methyl]-1-ethyl-2-phenyl-cyclopropanamine; trans-N-[(2-chloro-3-pyridyl)methyl]-1-ethyl-2-phenyl-cyclopropanamine; trans-N-[(2,2-dimethylchroman-6-yl)methyl]-1-ethyl-2-phenyl-cyclopropanamine; cis-1,2-diphenylcyclopropanamine; trans-1,2-diphenylcyclopropanamine; trans-1-ethyl-2-phenyl-cyclopropanamine; trans-2-(4-bromo-3-fluoro-phenyl)-1-ethyl-cyclopropanamine; trans 2-(3-bromophenyl)-1-phenethyl-cyclopropanamine; (1R,2S)-1,2-diphenylcyclopropanamine; (1S,2R)-1,2-diphenylcyclopropanamine; trans-2-(4-fluorophenyl)-1-(2-naphthylmethyl)cyclopropanamine; trans-2-(4-chlorophenyl)-1-(2-naphthylmethyl)cyclopropanamine; trans-2-(3-chlorophenyl)-1-(2-naphthylmethyl)cyclopropanamine; trans-2-(3-bromophenyl)-1-(2-naphthylmethyl)cyclopropanamine; trans-2-(4-chlorophenyl)-1-phenethyl-cyclopropanamine; trans-2-(4-fluorophenyl)-1-phenethyl-cyclopropanamine; trans-1-benzyl-2-(4-fluorophenyl)cyclopropanamine; trans-1-benzyl-2-(4-chlorophenyl)cyclopropanamine; trans 2-(4-bromophenyl)-1-phenethyl-cyclopropanamine; cis-1-ethyl-2-phenyl-cyclopropanamine; N-[4-(trans-2-amino-2-ethyl-cyclopropyl)phenyl]-3-[(1-methyl-4-piperidyl)amino]-4-phenyl-benzamide; 2-(4-benzyloxyphenyl)-1-(2-naphthylmethyl)cyclopropanamine; N-[4-trans-[2-amino-2-ethyl-cyclopropyl]phenyl]-2-(1-naphthyl)acetamide; N-[4-trans-[2-amino-2-ethyl-cyclopropyl]phenyl]-2-(4-nitrophenyl)acetamide; benzyl N-[4-[[4-[trans-2-amino-2-(2-naphthylmethyl)cyclopropyl]phenyl]carbamoyl]phenyl]carbamate; N-[4-(trans-2-amino-2-ethyl-cyclopropyl)phenyl]naphthalene-1-carboxamide; N-[4-[trans-2-amino-2-(2-naphthylmethyl)cyclopropyl]phenyl]naphthalene-2-carboxamide; N-[4-[trans-2-amino-2-(2-naphthylmethyl)cyclopropyl]phenyl]-4-phenyl-benzamide; N-[4-trans-2-amino-2-ethyl-cyclopropyl]phenyl]-2-(2-naphthyl)acetamide; N-[4-[trans-2-amino-2-phenyl-cyclopropyl]phenyl]benzamide; N-[4-[trans-2-amino-2-(2-naphthylmethyl)cyclopropyl]phenyl]-2-(1-naphthyl)acetamide; N-[3-[trans-2-amino-2-ethyl-cyclopropyl]phenyl]-2-(1-naphthyl)acetamide; N-[4-[trans-2-amino-2-ethyl-cyclopropyl]phenyl]-4-(3-furyl)benzamide; N-[4-[trans-2-amino-2-(2-naphthylmethyl)cyclopropyl]phenyl]-3-chloro-benzamide; N-[3-[trans-2-amino-2-ethyl-cyclopropyl]phenyl]-3-chloro-benzamide; N-[4-[trans-2-amino-2-(2-naphthylmethyl)cyclopropyl]phenyl]-3-phenyl-propanamide; N-[4-[trans-2-amino-2-(2-naphthylmethyl)cyclopropyl]phenyl]-3-phenyl-benzamide; N-[4-[trans-2-amino-2-ethyl-cyclopropyl]phenyl]-4-(2-oxooxazolidin-3-yl)benzamide; benzyl N-[3-[(4-[trans-2-amino-2-phenyl-cyclopropyl]phenyl)carbamoyl]phenyl]carbamate; N-[4-[trans-2-amino-2-phenyl-cyclopropyl]phenyl]naphthalene-2-carboxamide; N-[4-[trans-2-amino-2-ethyl-cyclopropyl]-2-fluoro-phenyl]benzamide; N-[4-[trans-2-amino-2-ethyl-cyclopropyl]-2-fluoro-phenyl]-4-phenyl-benzamide; N-[4-[trans-2-amino-2-ethyl-cyclopropyl]phenyl]-4-morpholino-benzamide; N-[4-[trans-2-amino-2-phenyl-cyclopropyl]phenyl]-4-phenyl-benzamide; N-[4-[trans-2-amino-2-phenyl-cyclopropyl]phenyl]naphthalene-1-carboxamide; N-[3-[trans-2-amino-2-ethyl-cyclopropyl]phenyl]-4-phenyl-benzamide; N-[3-[trans-2-amino-2-ethyl-cyclopropyl]phenyl]naphthalene-2-carboxamide; N-[3-[trans-2-amino-2-ethyl-cyclopropyl]phenyl]benzamide; N-[4-[trans-2-amino-2-ethyl-cyclopropyl]-2-fluoro-phenyl]-2-(1-naphthyl)acetamide; benzyl N-[4-[[4-[trans-2-amino-2-ethyl-cyclopropyl]-2-fluoro-phenyl]carbamoyl]phenyl]carbamate; N-[4-[trans-2-amino-2-ethyl-cyclopropyl]-2-fluoro-phenyl]-4-(4-methylpiperazin-1-yl)benzamide; N-[4-[trans-2-amino-2-ethyl-cyclopropyl]phenyl]-4-(4-methylpiperazin-1-yl)benzamide; N-[4-[trans-2-amino-2-ethyl-cyclopropyl]phenyl]-3-(2-oxooxazolidin-3-yl)benzamide; benzyl N-[5-[[4-[trans-2-amino-2-ethyl-cyclopropyl]phenyl]carbamoyl]-2-morpholino-phenyl]carbamate; N-[4-[trans-2-amino-2-ethyl-cyclopropyl]phenyl]-4-(1-methyl-4-piperidyl)benzamide; N-[4-(trans-2-amino-2-ethyl-cyclopropyl)phenyl]pyridine-4-carboxamide; N-[4-(trans-2-amino-2-ethyl-cyclopropyl]phenyl)-4-(4-pyridyl)benzamide; N-[4-(trans-2-amino-2-phenyl-cyclopropyl]phenyl]-3-chloro-benzamide; N-[4-(trans-2-amino-2-phenyl-cyclopropyl)phenyl]-2-(1-naphthyl)acetamide; N-[4-(trans-2-amino-2-phenyl-cyclopropyl)phenyl]-2-phenyl-acetamide; N-[4-(trans-2-amino-2-phenyl-cyclopropyl)phenyl]-3-phenyl-benzamide; N-[4-(trans-2-amino-2-phenyl-cyclopropyl)phenyl]-2-(2-naphthyl)acetamide; N-[4-(trans-2-amino-2-phenyl-cyclopropyl)phenyl]pyridine-4-carboxamide; N-[4-(trans-2-amino-2-phenyl-cyclopropyl]phenyl)-4-(1-methyl-4-piperidyl)benzamide; N-[4-(trans-2-amino-2-phenyl-cyclopropyl)phenyl]-4-(4-methylpiperazin-1-yl)benzamide; N-[4-(trans-2-amino-2-phenyl-cyclopropyl)phenyl]-3-(2-oxooxazolidin-3-yl)benzamide; N-[4-(trans-2-amino-2-phenethyl-cyclopropyl)phenyl]pyridine-4-carboxamide; N-[4-(trans-2-amino-2-phenethyl-cyclopropyl)phenyl]-4-phenyl-benzamide; N-[4-trans-2-amino-2-phenethyl-cyclopropyl)phenyl]benzamide; N-[4-(trans-2-amino-2-phenethyl-cyclopropyl)phenyl]-4-(1-methyl-4-piperidyl)benzamide; N-[4-(trans-2-amino-2-phenethyl-cyclopropyl)phenyl]-4-(4-methylpiperazin-1-yl)benzamide; benzyl N-[5-[[4-[trans-2-amino-2-ethyl-cyclopropyl]phenyl]carbamoyl]-2-(4-methylpiperazin-1-yl)phenyl]carbamate; N-[4-(trans-2-amino-2-phenethyl-cyclopropyl)phenyl]-3 -(2-oxooxazolidin-3-yl)benzamide; 4-[trans-2-amino-2-(2-naphthylmethyl)cyclopropyl]aniline; N-[4-(trans-2-amino-2-ethyl-cyclopropyl]phenyl)-4-(2-hydroxyethylamino)benzamide; benzyl N-[3-[1-[4-[2-amino-2-ethyl-cyclopropyl]phenyl]triazol-4-yl]phenyl]carbamate; trans-1-ethyl-2-[3-(4-phenyltriazol-1-yl)phenyl]cyclopropanamine; benzyl N-[3-[1-[4-[2-amino-2-(2-naphthylmethyl)cyclopropyl]phenyl]triazol-4-yl]phenyl]carbamate; trans-1-(2-naphthylmethyl)-2-[4-(4-phenyltriazol-1-yl)phenyl]cyclopropanamine; trans 1-ethyl-2-[2-(4-phenyltriazol-1 -yl)phenyl]cyclopropanamine; trans-1-benzyl-2-[4-(4-phenyltriazol-1-yl)phenyl]cyclopropanamine; N-[4-[(1S,2R)-2-amino-2-ethyl-cyclopropyl]phenyl]-4-phenyl-benzamide; N-[4-[(1R,2S)-2-amino-2-ethyl-cyclopropyl]phenyl]-4-phenyl-benzamide; trans 1-benzyl-2-(3-methoxyphenyl)cyclopropanamine; 1-[3-[(trans-2-amino-2-ethyl-cyclopropyl]phenyl]-3-phenyl-imidazolidin-2-one; trans-1-ethyl-2-[4-(4-phenyltriazol-1-yl)phenyl]cyclopropanamine; trans 1-[(benzylamino)methyl]-2-phenyl-cyclopropanamine; trans 1-[(cyclopropylamino)methyl]-2-phenyl-cyclopropanamine; trans 1-[(4-methylpiperazin-1-yl)methyl]-2-phenyl-cyclopropanamine; 5-[[[trans-1-methyl-2-phenyl-cyclopropyl]amino]methyl]pyrimidin-2-amine; trans-N-[(2-methoxy-3-pyridyl)methyl]-1-methyl-2-phenyl-cyclopropanamine; trans-N-(2,3-dihydro-1,4-benzodioxin-6-ylmethyl)-1-methyl-2-phenyl-cyclopropanamine; cis-N,1-dimethyl-2-phenyl-cyclopropanamine;2-[[trans-1,2-diphenylcyclopropyl]amino]-1-(4-methylpiperazin-1-yl)ethanone; 1-(4-methylpiperazin-1-yl)-2-[[trans-1-(2-naphthylmethyl)-2-phenyl-cyclopropyl]amino]ethanone; 2-[[(1R,2 S)-1-methyl-2-phenyl-cyclopropyl]amino]-1-(4-methylpiperazin-1-yl)ethanone; 2-[[(1R,2R)-1-methyl-2-phenyl-cyclopropyl]amino]-1-(4-methylpiperazin-1-yl)ethanone; 2-[[trans-1 -methyl-2-phenyl-cyclopropyl]amino]-1-(4-methylpiperazin-1-yl)ethanone; 2-[[cis-1-methyl-2-phenyl-cyclopropyl]amino]-1-(4-methylpiperazin-1-yl)ethanone; trans-N, 1-dimethyl-2-phenyl-cyclopropanamine; 2-[[trans-1-ethyl-2-phenyl-cyclopropyl]amino]-1-(1-piperidyl)ethanone;trans-1-ethyl-2-phenyl-N-[2-(1-piperidyl)ethyl]cyclopropanamine; 5-[[[trans-1-methyl-2-phenyl-cyclopropyl]amino]methyl]-1,3,4-oxadiazol-2-amine; trans-1-(4-nitrophenyl)-2-phenyl-cyclopropanamine; trans-2-(4-chlorophenyl)-1-phenyl-cyclopropanamine; trans-2-(4-bromophenyl)-1-phenyl-cyclopropanamine; N-[4-(trans-1-amino-2-phenyl-cyclopropyl]phenyl]acetamide; or a stereoisomer or pharmaceutically acceptable salt thereof.

In one embodiment, the LSD1 inhibitor is selected from a compound of Formula (III)

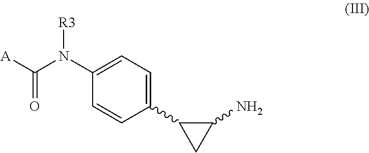

or an isomer, tautomer, racemic form, enantiomer, diastereomer, epimer, polymorph, solvate, mixtures thereof, pharmaceutically acceptable salt thereof, wherein:

A is R or CH(R₁)—NH—CO—R₂;
R and R₂ are selected from: alkyl, alkenyl, alkynyl, cycloalkyl, aryl, heteroaryl, heterocycloalkyl, cycloalkylalkyloxy, arylalkyloxy, heteroarylalkyloxy, heterocycloalkylalkyloxy, cycloalkylalkyl, arylalkyl, heteroarylalkyl, heterocycloalkylalkyl, cycloalkylalkylamino, arylalkylamino, heteroarylalkylamino, heterocycloalkylalkylamino;
R₁ is selected from: alkyl, alkenyl, alkynyl, cycloalkyl, aryl, heteroaryl, heterocycloalkyl, cycloalkylalkyl, arylalkyl, heteroarylalkyl, heterocycloalkylalkyl;
R₃ is H, C₁-C₆ alkyl.

In one embodiment, the LSD1 inhibitor is selected from a compound of Formula (III) that is: trans benzyl 4-(2-aminocyclopropyl)phenylcarbamate; trans N-(4-(2-aminocyclopropyl)phenyl)benzamide; trans N-(4-(2-aminocyclopropyl)phenyl)-1-naphthamide; trans N-(4-(2-aminocyclopropyl)phenyl)-2-naphthamide; trans N-(4-(2-aminocyclopropyl)phenyl)biphenyl-4-carboxamide; trans N-(4-(2-aminocyclopropyl)phenyl)-2-phenylacetamide; trans N-(4-(2-aminocyclopropyl)phenyl)-2-(naphthalen-1-yl)acetamide; trans N-(4-(2-aminocyclopropyl)phenyl)-2-(naphthalen-2-yl)acetamide; trans benzyl 1-(4-(2-aminocyclopropyl)phenylamino)-3-methyl-1-oxobutan-2-ylcarbamate; trans benzyl 1-(4-(2-aminocyclopropyl)phenylamino)-4-methyl-1-oxopentan-2- ylcarbamate; trans benzyl 1-(4-(2-aminocyclopropyl)phenylamino)-3-cyclohexyl-1-oxopropan-2-ylcarbamate; trans benzyl 2-(4-(2-aminocyclopropyl)phenylamino)-2-oxo-1-phenylethylcarbamate; trans benzyl 1-(4-(2-aminocyclopropyl)phenylamino)-1-oxo-3-phenylpropan-2-ylcarbamate; trans benzyl 1-(4-(2-aminocyclopropyl)phenylamino)-3-(4-bromophenyl)-1-oxopropan-2-ylcarbamate; trans benzyl 1-(4-(2-aminocyclopropyl)phenylamino)-3-(4-methoxyphenyl)-1-oxopropan-2-ylcarbamate; trans benzyl 1-(4-(2-aminocyclopropyl)phenylamino)-1-oxo-4-phenylbutan-2-ylcarbamate; trans benzyl 1-(4-(2-aminocyclopropyl)phenylamino)-1-oxo-3,3-diphenylpropan-2-ylcarbamate; trans benzyl 1-(4-(2-aminocyclopropyl)phenylamino)-3-(naphthalen-l-yl)-1-oxopropan-2-ylcarbamate; trans benzyl 1-(4-(2-aminocyclopropyl)phenylamino)-3-(naphthalen-2-yl)-1-oxopropan-2-ylcarbamate; trans benzyl 1-(4-(2-aminocyclopropyl)phenylamino)-4-(1H-indo1-3-yl)-1-oxobutan-2-ylcarbamate; trans benzyl 1-(4-(2-aminocyclopropyl)phenylamino)-4-(benzo[b]thiophen-3-yl)-1-oxobutan-2-ylcarbamate; trans 4-bromobenzyl 1-(4-(2-aminocyclopropyl)phenylamino)-1-oxo-3-phenylpropan-2-ylcarbamate; cis benzyl 1-(4-(2-aminocyclopropyl)phenylamino)-1-oxo-3-phenylpropan-2-ylcarbamate; trans N1-(4-(2-aminocyclopropyl)phenyl)-N8-hydroxyoctanediamide; trans benzyl 1-((4-(2-aminocyclopropyl)phenyl)(methyl)amino)-1-oxo-3-phenylpropan-2-ylcarbamate; trans N-(4-(2-aminocyclopropyl)phenyl)-2-(3-benzylureido)-3-phenylpropanamide or a isomer, tautomer, racemic form, enantiomer, diastereomer, epimer, polymorph, solvate, mixtures thereof, pharmaceutically acceptable salt thereof

In one embodiment, the LSD1 inhibitor is selected from: N1-((trans)-2-phenylcyclopropyl)cyclohexane-1,4-diamine; (cis)-N1-((1S,2R)-2-phenylcyclopropyl)cyclohexane-1,4-diamine; (trans)-N1-((1S,2R)-2-phenylcyclopropyl)cyclohexane-1,4-diamine; (cis)-N1-((1R,2S)-2-phenylcyclopropyl)cyclohexane-1,4-diamine; (trans)-N1-((1R,2S)-2-phenylcyclopropyl)cyclohexane-1,4-diamine; N1-((trans)-2-(thiazol-5-yl)cyclopropyl)cyclohexane-1,4-diamine; 1 -((trans)-2-(pyridin-3-yl)cyclopropyl)cyclohexane-1,4-diamine; N1-((trans)-2-(6-(3-(trifluoromethyl)phenyl)pyridin-3-yl)cyclopropyl)cyclohexane-1,4-diamine; N1-((trans)-2-(3′-(trifluoromethyl)-[1,1′-biphenyl]-4-y1)cyclopropyl)cyclohexane-1,4-diamine; N1-((trans)-2-(4-(benzyloxy)phenyl)cyclopropyl)cyclohexane-1,4-diamine; 4-(((trans)-2-(6-(3-(trifluoromethyl)phenyl)pyridin-3-yl)cyclopropyl)amino)cyclohexanol; 4-(((trans)-2-(6-(3-(trifluoromethyl)phenyl)pyridin-3-yl)cyclopropyl)amino)cyclohexanecarboxamide; N-(4-(((trans)-2-(6-(3-(trifluoromethyl)phenyl)pyridin-3-yl)cyclopropyl)amino)cyclohexyl)acetamide; (4-(((trans)-2-(6-(3-(trifiuoromethyl)phenyl)pyrldin-3 -yl ; (R)-1-(4-(((trans)-2-phenylcyclopropyl)amino)cyclohexyl)pyrrolidin-3-amine; N1-((trans)-2-(4′-chloro-[1,1′-biphenyl]-4-yl)cyclopropyl)cyclohexane-1,4-diamine; N1 -((trans)-2-(3 ‘-chloro-[ 1, l’-biphenyl]-4-yl)cyclopropyl)cyclohexane-1,4-diamine; 4′-((trans)-2-((4-aminocyclohexyl)amino)cyclopropyl)-[1,1′-biphenyl]-3-ol; N-(4′-((trans)-2-((4-aminocyciohexyl)amino)cyclopropyl)-[1,1′-biphenyl]-3-yl)methanesulfonamide; N1-((trans)-2-(4-((2-fluorobenzyl)oxy)phenyl)cyclopropyl)cyclohexane-1,4-diamine; N1-((trans)-2-(4-((3-fluorobenzyl)oxy)phenyl)cyclopropyl)cyclohexane-1,4-diamine; N1-((trans)-2-(4-((4-fluorobenzyl)oxy)phenyl)cyclopropyl)cyclohexane-1,4-diamine; N1-methyl-N4-((trans)-2-phenylcyclopropyl)cyclohexane-1,4-diamine; N1-methyl-N4-((trans)-2-(3′-(trifluoromethyl)-[1,1′-biphenyl]-4-yl)cyclopropyl)cyclohexane-1,4-diamine; N1-((trans)-2-(4-(benzyloxy)phenyl)cyclopropyl)-N4-methylcyclohexane-1,4-diamine; N1-((trans)-2-phenylcyclopropyl)cyclobutane-1,3-diamine; N1-((trans)-2-(3′-(trifluoromethyl)-[1,1′-biphenyl]-4-yl)cyclopropyl)cyclobutane-1,3-diamine; N1 -((trans)-2-(4-(benzyloxy)phenyl)cyclopropyl)cyclobutane-1,3-diamine; N1-((trans)-2-phenylcyclopropyl)-2,3-dihydro-1H-indene-1,3-diamine; N1-((trans)-2-(3′-(trifluoromethyl)-[1,1′-biphenyl]-4-yl)cyclopropyl)-2,3-dihydro-1H-indene-1,3-diamine; N1-((trans)-2-(4-(benzyloxy)phenyl)cyclopropyl)-2,3-dihydro-1H-indene-1,3-diamine; N1-((trans)-2-fluoro-2-phenylcyclopropyl)cyclohexane-1,4-diamine; N1-((1S,2S)-2-fluoro-2-phenylcyclopropyl)cyclohexane-1,4-diamine; N1-((1R,2R)-2-fluoro-2-phenylcyclopropyl)cyclohexane-1,4-diamine; 1-methyl-N4-((trans)-2-phenylcyclopropyl)cyclohexane-1,4-diamine; 4-(aminomethyl)-N-((trans)-2-phenylcyclopropyl)cyclohexanamine; N1-((trans)-2-phenylcyclopropyl)cyclohexane-1,3-diamine; N1-((cis)-2-phenylcyclopropyl)cyclohexane-1,4-diamine; tert-butyl (4-(((trans)-2-phenylcyclopropyl)amino)cyclohexyl)carbamate; 1-ethyl-3-(4-(((trans)-2-phenylcyclopropyl)amino)cyclohexyl)urea; 4-morpholino-N-((trans)-2-phenylcyclopropyl)cyclohexanamine; N1-((trans)-2-(4-bromophenyl)cyclopropyl)cyclohexane- 1,4-diamine; N1-{2-(o-tolyl)cyclopropyl)cyclohexane-1,4-diamine; N1-(2-(4-(trifluoromethyl)phenyl)cyclopropyl)cyclohexane-1,4-diamine; N1-(2-(4-methoxyphenyl)cyclopropyl)cyclohexane-1,4-diamine; 4-(2-((4-aminocyclohexyl)amino)cyclopropyl)phenol; N1-(2-(2-fluorophenyl)cyclopropyl)cyclohexane-1,4-diamine; N1-(2-(3 ,4-difluorophenyl)cyclopropyl)cyclohexane-1,4-diamine; N1-(2-(naphthalen-2-yl)cyclopropyl)cyclohexane-1,4-diamine; N1-(2-methyl-2-phenylcyclopropyl)cyclohexane- 1,4-diamine; (R)-1 -(4-(((trans)-2-(3 ‘-(trifluoromethyl)[1,1′-biphenyl]-4-yl)cyclopropyl)amino)cyclohexyl)pyrrolidin-3-amine; (cis)-N1-((1S,2R)-2-(3′-(trifluoromethyl)-[1,1′-biphenyl]-4-yl)cyclopropyl)cyclohexane-1,4-diamine; (trans)-N1-((1S,2R)-2-(3′-(trifiuoromethyl)-[1,1′-biphenyl]-4-yl)cyclo-propyl)cyclohexane-1,4-diamine; (cis)-N1-((1R,2S)-2-(3′-(trifluoromethyl)-[1,1’-biphenyl]-4-yl)cyclo-propyl)cyclohexane-1,4-diamine; (Trans)-N1-({1R,2S)-2-(3′-(trifluoromethyl)-[1,1′-biphenyl]-yl)cyclopropyl)cyclohexane-1,4-diamine; N1-((trans)-2-(4-cyclopropylphenyl)cyclopropyl)cyclohexane-1,4-diamine; N1-((trans)-2-(4-(pyridin-3-yl)phenyl)cyclopropyl)cyclohexane-1,4-diamine; N1-((trans)-2-(4-(1H-indazol-6-yl)phenyl)cyclopropyl)cyclohexane-1,4-diamine; N1-((trans)-2-(4-(1H-pyrazol-5-yl)phenyl)cyclopropyl)cyclohexane-1,4-diamine; 3-(5-((trans)-2-((4-aminocyclohexyl)amino)cyclopropyl)thiophen-2-yl)phenol; 3-(5-((trans)-2-((4-aminocyciohexyl)amino)cyclopropyl)thiazol-2-yl)phenol; 3-(5-((trans)-2-((4-aminocyclohexyl)amino)cyclopropyl)pyridin-2-yl)-5-methoxybenzonitrile; 5-5-((trans)-2-((4-aminocyclohexyl)amino)cyclopropyl)pyridin-2-yl)-2-methylphenol; N-(4′-((trans)-2-(4-aminocyclohexyl)amino)cyclopropyl)-6-methoxy-[1,1,-biphenyl]-3-yl)methanesulfonamide; N-(3-(5-((trans)-2-((4-aminocyclohexyl)amino)cyclopropyl)thiazol-2-yl)phenyl)-2-cyanobenzenesulfonamide; N-(4′-((trans)-2-((4-aminocyclohexyl)amino)cyclopropyl)[1,1′-biphenyl]-3-yl)-2-cyanobenzenesulfonamide; 6-amino-N-(4′-((trans)-2-((4-aminocyclohexyl)amino)cyclopropyl)-[1,1′-biphenyl]-3-yl)pyridine-3-sulfonamide; N-(4′-((trans)-2-((4-aminocyclohexyl)amino)cyclopropyl)-[1,1′-biphenyl]-3-yl)piperazine-1-sulfonamide; N1-((cis)-2-fluoro-2-phenylcyclopropyl)cyclohexane-1,4-diamine; N1-((trans)-2-(4-((3-(piperazin-1-yl)benzyl)oxy)phenyl)cyclopropyl)cyclohexane-1,4-diamine; N1-((trans)-2-(4-(pyridin-3-ylmethoxy)phenyl)cyclopropyl)cyclohexane-1,4-diamine; N1-((trans)-2-(6-((3-methylbenzyl)amino)pyridin-3-yl)cyclopropyl)cyclohexane-1,4-diamine; 3-((5-((trans)-2-((4-aminocyclohexyl)amino)cyclopropyl)pyridin-2-yl)aminobenzonitrile; N1-((trans)-2-(naphthalen-2-yl)cyclopropyl)cyclohexane-1,4-diamine; N1-((trans)-2-(o-tolyl)cyclopropyl)cyclohexane-1,4-diamine; N1-((trans)-2-(4-(trifluoromethyl)phenyl)cyclopropyl)cyclohexane-1,4-diamine; N1-((trans)-2-(4-methoxyphenyl)cyclopropyl)cyclohexane-1,4-diamine; N1-((trans)-2-(2-fluorophenyl)cyclopropyl)cyclohexane-1,4-diamine; N1-((trans)-2-(3,4-difluorophenyl)cyclopropyl)cyclohexane-1,4-diamine; N1-((trans)-2-methyl-2-phenylcyclopropyl)cyclohexane-1,4-diamine; (cis)-N1-((1S,2R)-2-(pyridin-3-yl)cyclopropyl)cyclohexane-1,4-diamine; (trans)-N1-((1R,2S)-2-(pyridin-3-yl)cyclopropyl)cyclohexane-1,4-diamine; (cis)-N1-((1R,2S)-2-(pyridin-3-yl)cyclopropyl)cyclohexane-1,4-diamine; (trans)-N1-((1S,2R)-2-(pyridin-3-yl)cyclopropyl)cyclohexane-1,4-diamine; (cis)-N1-((1S,2R)-2-phenylcyclopropyl)cyclobutane-1,3-diamine; trans N1-(1R,2S)-2-phenylcyclopropyl)cyclobutane-1,3-diamine; (cis)-N1-((1R,2S)-2-phenylcyclopropyl)cyclobutane-1,3-diamine; (trans)-N1-((1S,2R)-2-phenylcyclopropyl)cyclobutane-1,3-diamine; (cis)-N1-((1S,2R)-2-(3,4-difluorophenyl)cyclopropyl)cyclohexane-1,4-diamine; (trans)-N1-((1R,2S)-2-(3,4-difluorophenyl)cyclopropyl)cyclohexane-1,4-diamine; (cis)-N1-((1R,2S)-2-(3,4-difluorophenyl)cyclopropyl)cyclohexane-1,4-diamine; (trans)-N1-((1S,2R)-2-(3,4-difluorophenyl)cyclopropyl)cyclohexane-1,4-diamine; (cis)-N1-((1S,2R)-2-(naphthalen-2-yl)cyclopropyl)cyclohexane-1,4-diamine; (trans)-N1-((1R,2S)-2-(naphthalen-2-yl)cyclopropyl)cyclohexane-1,4-diamine; (cis)-1-((1R,2S)-2-(naphthalen-2-yl)cyclopropyl)cyclohexane-1,4-diamine; (trans)-N1-((1S,2R)-2-(naphthalen-2-yl)cyclopropyl)cyclohexane-1,4-diamine; (cis)-N1-((1S,2R)-2-(4-(1H-pyrazol-5-yl)phenyl)cyclopropyl)cyclohexane-1,4-diamine; (trans)-N1-((1R,2S)-2-(4-(1H-pyrazol-5-yl)phenyl)cyclopropyl)cyclohexane-1,4-diamine; (cis)-N1-((1R,2S)-2-(4-(1H-pyrazol-5-yl)phenyl)cyclopropyl)cyclohexane-1,4-diamine; (trans)-N1-((1S,2R)-2-(4-(1H-pyrazol-5-yl)phenyl)cyclopropyl)cyclohexane-1,4-diamine; N-(4′-((1R,2S)-2-(((cis)-4-aminocyclohexyl)amino)cyclopropyl)-[1,1′-biphenyl]-3-yl)piperazine-1-sulfonamide; N-(4′-((1S,2R)-2-(((trans)-4-aminocyclohexyl)amino)cyclopropyl)-[1,1′-biphenyl]-3-yl)piperazine-1-sulfonamide; N-(4′-((1S,2R)-2-(((cis)-4-aminocyclohexyl)amino)cyclopropyl)-[1,1′-biphenyl]-3-yl)piperazine-1-sulfonamide; N-(4′-((1R,2S)-2-(((trans)-4-aminocyciohexyl)amino)cyclopropyl)-[1,1′-biphenyl]-3-yl)piperazin; (cis)-N1-((1S,2R)-2-(4-((2-fluorobenzyl)oxy)phenyl)cyclopropyl)cyclohexane-1,4-diamine; (trans)-N1-((1R,2S)-2-(4-((2-fluorobenzyl)oxy)phenyl)cyclopropyl)cyclohexane-1,4-diamine; (cis)-N1-((1R,2S)-2-(4-((2-fluorobenzyl)oxy)phenyl)cyclopropyl)cyclohexane-1,4-diamine; (trans)-N1-((1S,2R)-2-(4-((2-fluorobenzyl)oxy)phenyl)cyclopropyl)cyclohexane-1,4-diamine; or a pharmaceutically acceptable salt or solvate thereof.

In one embodiment, the LSD1 inhibitor is selected from: 1,1-Dimethylethyl 4-({[trans-2-phenylcyclopropyl]amino}methyl)-1-piperidinecarboxylate; 1,1-Dimethylethyl 4-({[(1R,2S)-2-phenylcyclopropyl]amino}methyl)-1-piperidinecarboxylate; 1,1-Dimethylethyl 4-({[(1S,2R)-2-phenylcyclopropyl]amino}methyl)-1-piperidinecarboxylate; [trans-2-Phenylcyclopropyl]{[1-(phenylmethyl)-4-piperidinyl]methyl}amine; N-Phenyl-4-(((trans-2-phenylcyclopropyl)amino)methyl)piperidine-1-carboxamide; Phenyl(4-(((trans-2-phenylcyc opropyl)amino)methyl)piperidin-1-yl)methanone; 1-(4-(((trans-2-Phenylcyclopropyl)amino)methyl)piperidin-1-yl)ethanone; Benzyl 4-(((trans-2-phenylcyclopropyl)amino)methyl)piperidine-1-carboxylate; 1,1-Dimethylethyl 4-({[trans-2-phenylcyclopropyl]amino}methyl)hexahydro-1H-azepine-1-carboxylate 2-(4-(((trans-2-phenylcyclopropyl)amino)methyl)piperidin-1-yl)acetic acid; 4-{[(3R)-3-({[(1R,2S)-2-Phenylcyclopropyl]amino}methyl)-1-pyrrolidinyl]methyl}benzoic acid; 4-{[(3S)-3-({[(1R,2 S)-2-Phenylcyclopropyl]amino}methyl)-1-pyrrolidinyl]methyl}benzoic acid; 4-{3-[4-({[(1R,2S)-2-Phenylcyclopropyl]amino}methyl)-1-piperidinyl]propyl}benzoic acid; trans-2-Phenyl-N-((1-(pyridin-4-ylmethyl)piperidin-4-yl)methyl)cyclopropanamine; trans-N-((1-(2-Fluorobenzyl)piperidin-4-yl)methyl)-2-phenylcyclopropanamine; trans-N-((1-(3-Fluorobenzyl)piperidin-4-yl)methyl)-2-phenylcyclopropanamine; trans-N-((1-(4-Fluorobenzyl)piperidin-4-yl)methyl)-2-phenylcyclopropanamine; trans-N-((1-(2,4-Difluorobenzyl)piperidin-4-yl)methyl)-2-phenylcyclopropanamine; Ethyl 4-((4-(((trans-2-phenylcyclopropyl)amino)methyl)piperidin-1-yl)methyl)benzoate; trans-N-((1-(4-(Methyl sulfonyl)benzyl)piperidin-4-yl)methyl)-2-phenylcyclopropanamine; 2-((4-(((trans-2-Phenylcyclopropyl)amino)methyl)piperidin-1-yl)methyl)benzonitrile; trans-2-Phenyl-N-((1-(2-(trifluoromethyl)benzyl)piperidin-4-yl)methyl)cyclopropanamine; trans-N-((1-((5-Methylisoxazol-3-yl)methyl)piperidin-4-yl)methyl)-2-phenylcyclopropanamine; trans-N-((1-((1H-Pyrazol-4-yl)methyl)piperidin-4-yl)methyl)-2-phenylcyclopropanamine; N-(4-((4-(((trans-2-Phenylcyclopropyl)amino)methyl)piperidin-1-yl)methyl)phenyl)acetamide; 4-((4-(((trans-2-Phenylcyclopropyl)amino)methyl)piperidin-1-yl)methyl)benzo[c][1,2]oxaborol-1(3H)-ol; 5((4-(((trans-2-Phenylcyclopropyl)amino)methyl)piperidin-1-yl)methyl)benzo[c][1,2]oxaborol-1(3H)-ol; (4-((4-(((trans-2-phenylcyclopropyl)amino)methyl)piperidin-1-yl)methyl)phenyl)boronic acid; 2-((4-(((trans-2-Phenylcyclopropyl)amino)methyl)piperidin-1-yl)methyl)benzoic acid; 3-((4-(((trans-2-Phenylcyclopropyl)amino)methyl)piperidin-1-yl)-methyl)benzoic acid; 4-((4-(((trans-2-(4-Bromophenyl)cyclopropyl)amino)methyl)piperidin-1-yl)methyl)benzoic acid; 4-((4-(((trans-2-(4-Chlorophenyl)cyclopropyl)amino)methyl)piperidin-1-yl)methyl)benzoic acid; 4-((4-(((trans-2-(3,4-Dichlorophenyl)cyclopropyl)amino)methyl)piperidin-1-yl)methyl)benzoic acid; 4-((4-(((trans-2-(4-(Trifluoromethyl)phenyl)cyclopropyl)amino)methyl)piperidin-1-yl)methyl)benzoic acid; 4-((4-(((trans-2-(3,4-Dimethoxyphenyl)cyclopropyl)amino)methyl)piperidin-1-yl)methyl)benzoic acid; 4-((4-(((trans-2-(4-Acetamidophenyl)cyclopropyl)amino)methyl)piperidin-1-yl)methyl)benzoic acid; 4-((4-(((trans-2-(4-Benzamidophenyl)cyclopropyl)amino)methyl)piperidin-1-yl)methyl)benzoic acid; trans-2-Phenyl-N-((1-phenylpiperidin-4-yl)methyl)cyclopropanamine; Ethyl 4-(((trans-2-phenylcyclopropyl)amino)methyl)piperidine-1-carboxylate; trans-4-((4-(((trans-2-Phenylcyclopropyl)amino)methyl)piperidin-1-yl)methyl)cyclohexanecarboxylic acid; (trans)-N-((1-(Methylsulfonyl)piperidin-4-yl)methyl)-2-phenylcyclopropanamine; N-ethyl-4-((((trans)-2-phenylcyclopropyl)amino)methyl)piperidine-1-carboxamide; N-cyclopropyl-4-((((trans)-2-phenylcyclopropyl)amino)methyl)piperidine-1-carboxamide; N,N-dimethyl-4-((((trans)-2-phenylcyclopropyl)amino)methyl)piperidine-1-carboxamide, (4-((((trans)-2-phenylcyclopropyl)amino)methyl)piperidin-1-yl)(pyrrolidin-1-yl)methanone; trans-N-((1-((cyclopropylsulfonyl)piperidin-4-yl)methyl)-2-phenylcyclopropanamine; trans-N-((1-((isopropylsulfonyl)piperidin-4-yl)methyl)-2-phenylcyclopropanamine; trans-N-((1-(3,5-dimethylisoxazol-4-yl)sulfonyl)piperidin-4-yl)methyl)-2-phenylcyclopropanamine; trans-N-((1-((1,2-dimethyl-1H-imidazol-4-yl)sulfonyl)piperidin-4-yl)methyl)-2-phenylcyclopropanamine; (trans)-2-Phenyl-N-(2-(1-(pyridin-2-yl)piperidin-4-yl)ethyl)cyclopropanamine; 6-(4-(2-(((trans)-2-Phenylcyclopropyl)amino)ethyl)piperidin-1-yl) nicotinic acid; trans-2-phenyl-N-(2-(1-(pyridin-4-yl)piperidin-4-yl)ethyl)cyclopropanamine; trans-2-phenyl-N-(2-(1-(pyrimidin-4-yl)piperidin-4-yl)ethyl)cyclopropanamine; trans-2-phenyl-N-(2-(1-phenylpiperidin-4-yl)ethyl)cyclopropanamine; trans-2-phenyl-N-(2-(1-(pyridin-3-yl)piperidin-4-yl)ethyl)cyclopropanamine; trans-2-phenyl-N-(2-(1-(pyrimidin-2-yl)piperidin-4-yl)ethyl)cyclopropan-amine; 3-Cyano-4-((4-((((trans)-2-phenylcyclopropyl)amino)methyl)piperidin-1-yl)methyl)benzoic acid; 2-fluoro-4-((4-((((trans)-2-phenylcyclopropyl)amino)methyl)piperidin-1-yl)methyl)benzoic acid; 3-fluoro-4-((4-((((trans)-2-phenylcyclopropyl)amino)methyl)piperidin-1-yl-)methyl)benzoic acid; 3-chloro-4-((4-((((trans)-2-phenylcyclopropyl)amino)methyl)piperidin-1-yl-)methyl)benzoic acid; 3-methoxy-4-((4-((((trans)-2-phenylcyclopropyl)amino)methyl)piperidin-1-yl)methyl)benzoic acid; 2-chloro-4-((4-((((trans)-2-phenylcyclopropyl)amino)methyl)piperidin-1-yl)methyl)benzoic acid; 4-(3-(4-(Cyano(((1R,2S)-2-phenylcyclopropyl)amino)methyl)piperidin-1-yl)propyl)benzoic acid; 4-{3-[4-({[(trans))-2-phenylcyclopropyl]amino}methyl)-1-piperidinyl]propyl}benzoic acid; 4-(4-(4-((((1R,2S)-2-Phenylcyclopropyl)amino)methyl)piperidin-1-yl)butyl)benzoic acid; 4-(4-(4-(Cyano(((1R,2S)-2-phenylcyclopropyl)amino)methyl)piperidin-1-yl)butyl)benzoic acid; 4-(2-(4-((((trans)-2-Phenylcyclopropyl)amino)methyl)piperidin-1-yl)ethyl)benzoic acid; 4-(2-(4-((((1R,2S)-2-phenylcyclopropyl)amino)methyl)piperidin-1-yl)ethyl)benzoic acid; 6-((4-((((trans)-2-Phenylcyclopropyl)amino)methyl)piperidin-1-yl)methyl)-2-naphthoic acid; 6-((4-((((1R,2S)-2-Phenylcyclopropyl)amino)methyl)piperidin-1-yl)methyl)-2-naphthoic acid; (trans)-N-((1-(4-(1H-Tetrazol-5-yl)benzyl)piperidin-4-yl)methyl)-2-phenylcyclopropanamine; 2-(4-((4-((((trans)-2-Phenylcyclopropyl)amino)methyl)piperidin-1-yl)methyl)benzamido)acetic acid; N-(4-((4-((((trans)-2-Phenylcyclopropyl)amino)methyl)piperidin-1-yl)methyl)phenyl)methanesulfonamide; (trans)-N-((1-(3-(1H-Tetrazol-5-yl)propyl)piperidin-4-yl)methyl)-2-phenylcyclopropanamine; 4-((4-(2-(((trans)-2-Phenylcyclopropyl)amino)ethyl)piperidin-1-yl)methyl)-benzoic acid; 6-((4-((((trans)-2-Phenylcyclopropyl)amino)methyl)piperidin-1-yl)methyl)nicotinic acid; 2-(4-((4-((((trans)-2-Phenylcyclopropyl)amino)methyl)piperidin-1-yl)methyl)phenyl)acetic acid; 2-((4-((((trans)-2-Phenylcyclopropyl)amino)methyl)piperidin-1-yl)methyl)o- xazole-4-carboxylic acid; 2-(4-((4-((((trans)-2-phenylcyclopropyl)amino)methyl)piperidin-1-yl)methyl)phenoxy)acetic acid; N-(Methylsulfonyl)-4-((4-((((trans)-2-phenylcyclopropyl)amino)methyl)piperidin-1-yl)methyl)benzamide; 4-((4-((((trans)-2-(4-Iodophenyl)cyclopropyl)amino)methyl)piperidin-1-yl)methyl)benzoic acid; 4-((trans)-2-(((1-Benzylpiperidin-4-yl)methyl)amino)cyclopropyl)benzoic acid; 4-((4-((((trans)-2-(4-(1-Methyl-1H-pyrazol-4-yl)phenyl)cyclopropyl)amino)methyl)piperidin-1-yl)methyl)benzoic acid; 4-((4-((((trans)-2-(4-Cyclopropylphenyl)cyclopropyl)amino)methyl)piperidin-1-yl)methyl)benzoic acid; 2-Chloro-4-((4-((((1R,2S)-2-phenylcyclopropyl)amino)methyl)piperidin-1-yl)methyl)benzoic acid; 3-(3-(4-((((trans)-2-Phenylcyclopropyl)amino)methyl)piperidin-1-yl)propyl)benzoic acid; or 2-(4-((4-((((1R,2S)-2-Phenylcyclopropyl)amino)methyl)piperidin-1-yl)methyl)phenyl)acetic acid or a pharmaceutically acceptable salt or solvate thereof.

In one embodiment, the LSD1 inhibitor, which may also be referred to as a KDM1A inhibitor, is selected from the group consisting of: tranylcypromine;

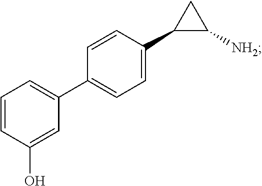 [SMILES: N[C@H]1C[C@@H]1c1ccc(-c2cccc(O)c2)cc1]

(R)-4-[5-(Pyrrolidin-3-ylmethoxy)-2-p-tolyl-pyridin-3-yl]-benzonitrile;
1 -(4-methyl-1-piperazinyl)-2-[[(1R*,2S*)-2-[4-phenylmethoxy)phenyl] cyclopropyl]amino] ethanone;

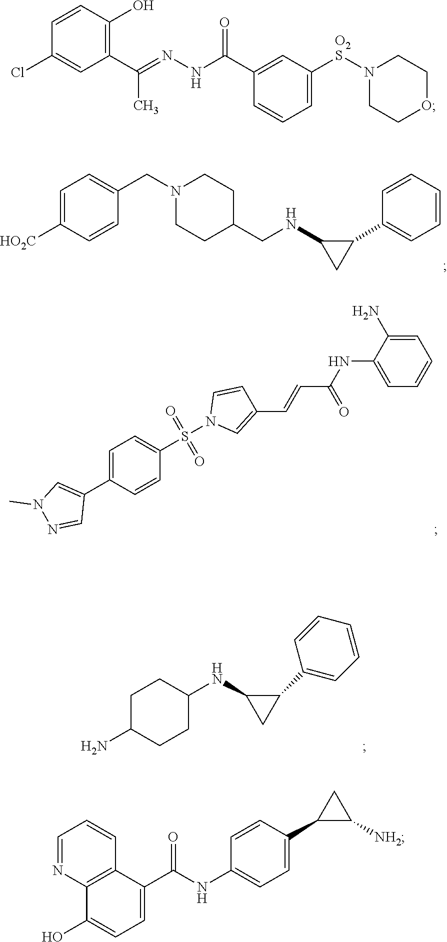

N-[4-[trans-2-aminocyclopropyl]phenyl]-4-(4-methylpiperazin-1-yl)benzamide; and a pharmaceutically acceptable salt thereof. (Maes, T. et al. Current Opinion in Phamacol. 2015, 23: 52-60).

In one embodiment, the LSD1 inhibitor is N-[4-[trans-2-aminocyclopropyl]phenyl]-4-(4-methylpiperazin-1-yl)benzamide, or a pharmaceutically acceptable salt thereof.

In one aspect, the disclosure relates to a pharmaceutical composition in the form of tablets, capsules, oral preparations, powders, granules, pills, injectable or infusible liquid, solutions, suspensions, emulsions, suppositories, ointments, creams, lotions, lozenges, chews, gels, pastes, multi- and nano-particulates, gels, solid solutions, liposomes, nanoparticles, films, ovules, sprays, injectables, and liquid formulations or transdermal delivery devices. A reference for the formulations is the book by Remington (“Remington: The Science and Practice of Pharmacy”, Lippincott Williams & Wilkins, 2000).

Compounds and/or compositions of the application may be administered to a subject in a total daily dose of, for example, from 0.001 to 1000 mg/kg body weight daily. Dosage unit compositions may contain such amounts of submultiples thereof to make up the daily dose. The determination of optimum dosages for a particular subject is well known to one skilled in the art.

Any of the compounds, combinations, pharmaceutical compositions, and/or dosage forms described herein can be administered to the subject via an oral, topical, intravenous, inhalational, otic, intramucosal, intraarterial, intraocular, intraperitoneal, intrathecal, intraventricular, intraurethral, intrasternal, intracranial, intramuscular, and/or subcutaneous route of administration.

Any of the compounds, combinations, pharmaceutical compositions, and/or dosage forms described herein can be administered to the subject on a daily (e.g., 1, 2, or 3 times daily), weekly (e.g., 1, 2, 3, 4, or 5 times weekly), or monthly basis (e.g., 1, 2, 3, 4, 5, 6, 7, 8, 9, or 10 times monthly). Determination of the appropriate dosing schedule is within the routine level of skill in the art.

Any of the compounds, combinations, pharmaceutical compositions, and/or dosage forms described herein may be formulated with pharmaceutically acceptable carriers or diluents as well as any other known adjuvants and excipients in accordance with conventional techniques such as those disclosed in Remington: The Science and Practice of Pharmacy, 21st Edition, 2000, Lippincott Williams & Wilkins, which is incorporated herein in its entirety.

As is common practice, the compositions are normally accompanied by written or printed instructions for use in the treatment in question.

Another aspect of the disclosure is a kit comprising an LSD1 inhibitor, combination or pharmaceutical composition as defined herein and at least one therapeutic agent selected from the group consisting of: histone deacetylase inhibitors, retinoid receptor modulators, antiproliferative/antineoplastic agents, cytostatic agents, agents which inhibit cancer cell invasion, inhibitors of growth factor function, antiangiogenic agents, proteasome inhibitors, HSP90 inhibitors, Selective COX-2 inhibitors and chemotherapeutic agents.

Optionally, the compound of the disclosure and the at least one therapeutic agent are in separated containers.

Also, various inventive concepts may be embodied as one or more methods or pharmaceutical compositions for use, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All publications, patent applications, patents, and other references mentioned herein are incorporated by reference in their entirety.

Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

All definitions, as defined and used herein, should be understood to control over dictionary definitions, definitions in documents incorporated by reference, and/or ordinary meanings of the defined terms.

The phrase “pharmaceutically acceptable excipient” refers to any of the following classes of ingredients: fillers, binders, lubricants, disintegrating agents, glidants (e.g., silicon dioxide), flavoring agents and colorants. Suitable binders include, e.g., microcrystalline cellulose (e.g., Avicel PH200 LM, PH112, PH101, PH102, PH103, PH113, PH105, PH200, DG), mannitol, dicalcium phosphate, dicalcium phosphate anhydrous, povidone, lactose, glucose, starch, gelatin, acacia gum, tragacanth gum, sodium alginate, carboxymethylcellulose, polyethylene glycol, waxes or the like. Lubricants include, e.g., glyceryl dibehenate, hydrogenated vegetable oil, sodium oleate, sodium stearate, magnesium stearate, silicon dioxide, sodium benzoate, sodium acetate, sodium chloride or the like. Other excipients include, e.g., starch, methyl cellulose, agar, bentonite, xanthan gum, sodium starch glycolate, crospovidone, croscarmellose sodium or the like. Additional excipients for capsules include macrogols or lipids and/or any other excipients known in the art.

Any of the compositions or pharmaceutical compositions described herein may be formulated with pharmaceutically acceptable carriers or diluents as well as any other known adjuvants and excipients in accordance with conventional techniques such as those disclosed in Remington: The Science and Practice of Pharmacy, 21s^{t} Edition, 2000, Lippincott Williams & Wilkins, which is incorporated herein in its entirety.

The term “about,” as used herein, and unless explicitly stated otherwise, refers to a recited value +/−10%, +/−5%, +/−2.5%, +/−1%, or +/−0.5%. For example, “about” may refer to a recited value +/−5%.

The term, “subject” as used herein refers to a human or non-human, i.e., a patient. In one embodiment, the subject is a mammal. In one embodiment, the subject is a human.

The phrase, “therapeutically effective amount” or “effective amount” as used herein indicates an amount necessary to administer to a subject, or to a cell, tissue, or organ of a subject, to achieve a therapeutic effect, such as an ameliorating or alternatively a curative effect.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, phrases containing the term “and/or” such as “A, B and/or C” refer to any of the following: A only; B only; C only; A and B; A and C; B and C; A, B and C.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for treating a subject having cancer, which comprises
a) administering to the subject an effective amount of a CDK2 inhibitor; and
b) administering to the subject an effective amount of a LSD1 inhibitor;
wherein the CDK2 inhibitor is administered before, concurrently, or after the LSD1 inhibitor.

2. The method of any claim 1, wherein the CDK2 inhibitor is administered about 24 hours, about 48 hours, about 72 hours, or about 1 week before the LSD1 inhibitor.

3. The method of claim 1, wherein the CDK2 inhibitor is selected from the group consisting of:
Milciclib, Dinaciclib, AG-024322, 2-Hydroxybohemine, Aloisine A, AZD 5438, BMS-265246, Butyrolactone I, CDK2 Inhibitor II (CAS # 222035-13-4), CDK2 Inhibitor IV, NU6140 (CAS # 444723-13-1), CYC-065, NU2058 (CAS # 161058-83-9), NU6102 (CAS # 444722-95-6), Olomoucine, PHA-793887 (CAS # 718630-59-2), Roscovitine (seliciclib), SCH 900776 (CAS # 891494-63-6), SNS-032 (BMS-387032), SU9516 (CAS # 377090-84-1), WHI-P180 (CAS # 21 1555-08-7), or any pharmaceutically acceptable salt thereof, and any combination thereof.

4. The method of claim 1, wherein the LSD1 inhibitor is selected from the group consisting of a compound of Formula (I), Formula (Ia), Formula (II), Formula (III), or any other compound indicated as an LSD1 inhibitor, including: MC2580, DDP38003, tranylcypromine, (R)-4-[5-(Pyrrolidin-3-ylmethoxy)-2-p-tolyl-pyridin-3-yl]-benzonitrile, 1-(4-methyl-1-piperazinyl)-2-[[(1R*,2S*)-2-[4-phenylmethoxy)phenyl]cyclopropyl]amino]ethanone, N-[4-[trans-2-aminocyclopropyl]phenyl]-4-(4-methylpiperazin-1-yl)benzamide, 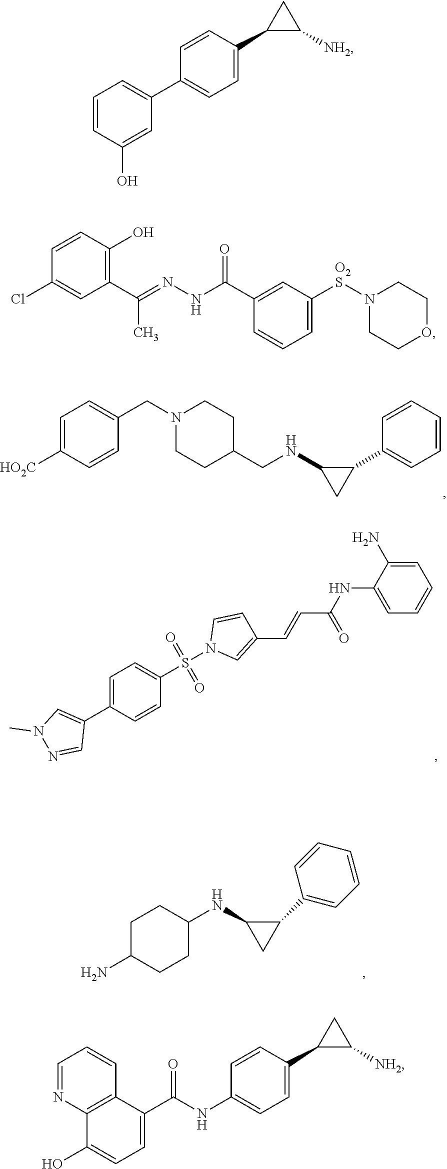
or a stereoisomer or a pharmaceutically acceptable salt thereof.

5. The method of claim 1, wherein the cancer is selected from the group consisting of leukemia (including acute promyelocytic leukemia and acute myeloid leukemia), prostate cancer, breast cancer, lung cancer (including small cell lung cancer or small cell lung carcinoma), colorectal cancer, melanomas, bladder cancer, brain/CNS cancer, cervical cancer, esophageal cancer, gastric cancer, head/neck cancer, kidney cancer, liver cancer, lymphomas, ovarian cancer, pancreatic cancer, and sarcomas.

6. The method of claim 1, wherein the cancer is:
a) a solid tumor;
b) a blood tumor; or
c) L SD1-inhibitor-resistant cancer.

7. The method of claim 6, wherein the LSD1-inhibitor-resistant cancer comprises cancerous cells having a reduced level of p21 expression or a loss of p21 function as compared to cancerous cells that are sensitive to LSD1 inhibitors.

8. A method for treating a subject having LSD1-inhibitor-resistant cancer by sensitizing cells of the cancer to LSD1 inhibitors, which comprises
a) administering to the subject an effective amount of a CDK2 inhibitor; and
b) administering to the subject an effective amount of a LSD1 inhibitor;
wherein the CDK2 inhibitor is administered before, concurrently, or after the LSD1 inhibitor and wherein the method optionally comprises a step of evaluating the cancer to predict resistance to LSD1 inhibitors prior to administration of the CDK2 inhibitor.

9. The method of claim 8, wherein the CDK2 inhibitor is administered about 24 hours, about 48 hours, about 72 hours, or about 1 week before the LSD1 inhibitor.

10. The method of claim 8, wherein the CDK2 inhibitor is selected from the group consisting of:
Milciclib, Dinaciclib, AG-024322, 2-Hydroxybohemine, Aloisine A, AZD 5438, BMS-265246, Butyrolactone I, CDK2 Inhibitor II (CAS # 222035-13-4), CDK2 Inhibitor IV, NU6140 (CAS # 444723-13-1), CYC-065, NU2058 (CAS # 161058-83-9), NU6102 (CAS # 444722-95-6), Olomoucine, PHA-793887 (CAS # 718630-59-2), Roscovitine (seliciclib), SCH 900776 (CAS # 891494-63-6), SNS-032 (BMS-387032), SU9516 (CAS # 377090-84-1), WHI-P180 (CAS # 21 1555-08-7), or any pharmaceutically acceptable salt thereof, and any combination thereof.

11. The method of claim 8, wherein the LSD1 inhibitor is selected from the group consisting of a compound of Formula (I), Formula (Ia), Formula (II), Formula (III), or any other compound indicated as an LSD1 inhibitor, including: MC2580, DDP38003, tranylcypromine, (R)-4-[5-(Pyrrolidin-3-ylmethoxy)-2-p-tolyl-pyridin-3-yl]-benzonitrile, 1-(4-methyl-1-piperazinyl)-2-[[(1R*,2S*)-2-[4-phenylmethoxy)phenyl]cyclopropyl]amino]ethanone, N-[4-[trans-2-aminocyclopropyl]phenyl]-4-(4-methylpiperazin-1-yl)benzamide, 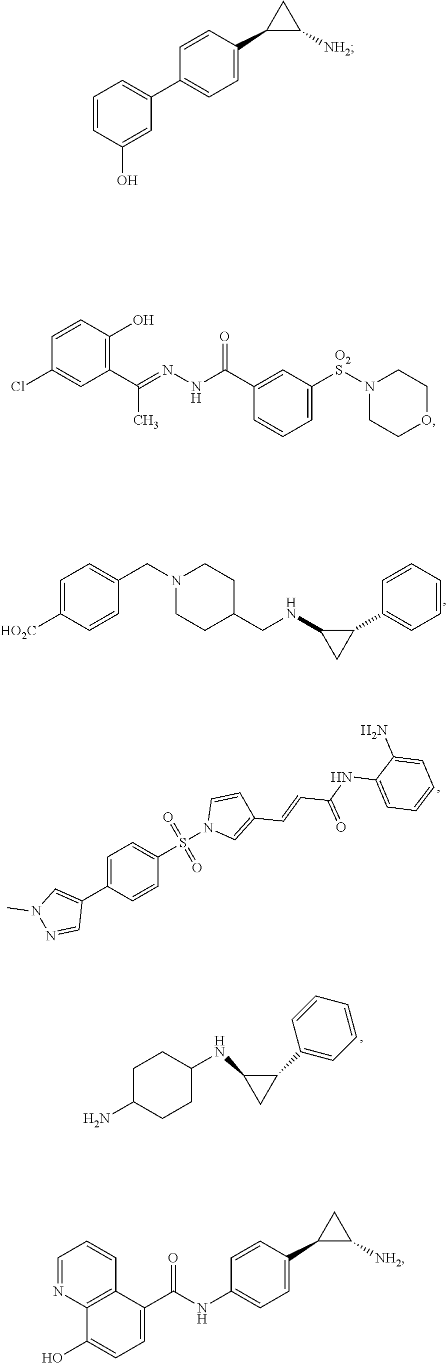
or a stereoisomer or a pharmaceutically acceptable salt thereof.

12. The method of claim 8, wherein the LSD1-inhibitor-resistant cancer comprises cancerous cells having a reduced level of p21 expression or a loss of p21 function as compared to cancerous cells that are sensitive to LSD1 inhibitors.

13. The method of claim 8, wherein the LSD1-inhibitor-resistant cancer is selected from the group consisting of leukemia (including acute promyelocytic leukemia and acute myeloid leukemia), prostate cancer, breast cancer, lung cancer (including small cell lung cancer or small cell lung carcinoma), colorectal cancer, melanomas, bladder cancer, brain/CNS cancer, cervical cancer, esophageal cancer, gastric cancer, head/neck cancer, kidney cancer, liver cancer, lymphomas, ovarian cancer, pancreatic cancer, and sarcomas.

14. The method of claim 8, wherein the LSD1-inhibitor-resistant cancer is a solid tumor or blood tumor.

15. A pharmaceutical composition for treating or ameliorating the effects of a cancer in a subject in need thereof, comprising
a) a therapeutically-effective amount of an LSD1 inhibitor;
b) a therapeutically-effective amount of a CDK2 inhibitor; and
c) a pharmaceutically-acceptable excipient;
wherein the LSD1 inhibitor and the CDK2 inhibitor are co-administered to the subject or are administered to the subject serially; and
wherein the LSD1 inhibitor and the CDK2 inhibitor are in a unified dosage form or in separate dosage forms.

16. The pharmaceutical composition of claim 15, wherein the CDK2 inhibitor is administered about 24 hours, about 48 hours, about 72 hours, or about 1 week before the LSD1 inhibitor.

17. The pharmaceutical composition of claim 15, wherein the CDK2 inhibitor is selected from the group consisting of: Milciclib, Dinaciclib, AG-024322, 2-Hydroxybohemine, Aloisine A, AZD 5438, BMS-265246, Butyrolactone I, CDK2 Inhibitor II (CAS # 222035-13-4), CDK2 Inhibitor IV, NU6140 (CAS # 444723-13-1), CYC-065, NU2058 (CAS # 161058-83-9), NU6102 (CAS # 444722-95-6), Olomoucine, PHA-793887 (CAS # 718630-59-2), Roscovitine (seliciclib), SCH 900776 (CAS # 891494-63-6), SNS-032 (BMS-387032), SU9516 (CAS # 377090-84-1), WHI-P180 (CAS # 21 1555-08-7), or any pharmaceutically acceptable salt thereof, and any combination thereof.

18. The pharmaceutical composition of claim 15, wherein the LSD1 inhibitor is selected from the group consisting of a compound of Formula (I), Formula (Ia), Formula (II), Formula (III), or any other compound indicated as an LSD1 inhibitor, including: MC2580, DDP38003, tranylcypromine, (R)-4-[5-(Pyrrolidin-3-ylmethoxy)-2-p-tolyl-pyridin-3-yl]-benzonitrile, 1-(4-methyl-1-piperazinyl)-2-[[(1R*,2S*)-2-[4-phenylmethoxy)phenyl]cyclopropyl]amino]ethanone, N-[4-[trans-2-aminocyclopropyl]phenyl]-4-(4-methylpiperazin-1-yl)benzamide, 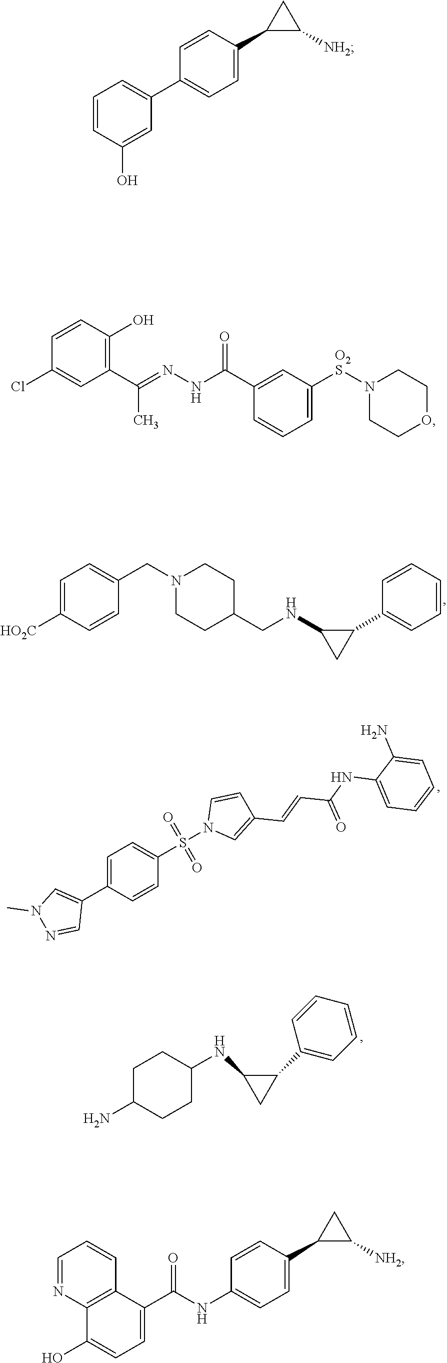
or a stereoisomer or a pharmaceutically acceptable salt thereof.

19. The pharmaceutical composition of claim 15, wherein the cancer is selected from the group consisting of leukemia (including acute promyelocytic leukemia and acute myeloid leukemia), prostate cancer, breast cancer, lung cancer (including small cell lung cancer or small cell lung carcinoma), colorectal cancer, melanomas, bladder cancer, brain/CNS cancer, cervical cancer, esophageal cancer, gastric cancer, head/neck cancer, kidney cancer, liver cancer, lymphomas, ovarian cancer, pancreatic cancer, and sarcomas.

20. The pharmaceutical composition of claim 15, wherein the cancer is:
a) a solid tumor;
b) a blood tumor; or
c) LSD1-inhibitor-resistant cancer;
wherein the LSD1-inhibitor-resistant cancer comprises cancerous cells having a reduced level of p21 expression or a loss of p21 function as compared to cancerous cells that are sensitive to LSD1 inhibitors.
